(19) United States
(12) Patent Application Publication
(10) Pub. No.: US 2022/0395487 A1
(43) Pub. Date: Dec. 15, 2022

(54) RIG-I INNATE IMMUNE RECEPTOR ANTAGONISTS AND METHODS OF USING SAME

(71) Applicant: YALE UNIVERSITY, New Haven, CT (US)

(72) Inventors: Anna Marie Pyle, Guilford, CT (US); David Rawling, Salt Lake City, UT (US); Erik G. Jagdmann, Holly Springs, NC (US); Olga Potapova, Cheshire, CT (US)

(21) Appl. No.: 17/773,889

(22) PCT Filed: Nov. 4, 2020
(86) PCT No.: PCT/US2020/058803

§ 371 Date: May 3, 2022

Related U.S. Application Data
(60) Provisional application No. 62/930,025, filed on Nov. 4, 2019.

(51) Int. Cl. A61K 31/403; A61K 31/4184; A61P 37/00

(52) U.S. Cl. CPC A61K 31/403; A61K 31/4184; A61P 37/00

(57) ABSTRACT

The present disclosure provides certain RIG-I antagonists. In certain embodiments, the antagonists of the disclosure can be used to treat or prevent a disease or disorder in a subject.

### CROSS-REFERENCE TO RELATED APPLICATIONS

This application claims priority under 35 U.S.C. § 119(e) to U.S. Provisional Patent Application No. 62/930,025, filed Nov. 4, 2019, which is incorporated herein by reference in its entirety.

### STATEMENT REGARDING FEDERALLY SPONSORED RESEARCH

This invention was made with government support under AI089826 awarded by National Institutes of Health. The government has certain rights in the invention.

### BACKGROUND

Vertebrate organisms express a diversity of innate immune receptor proteins that function as biosensors for the detection and response to infection. All types of pathogens, including viruses, bacteria and eukaryotic parasites, introduce foreign molecules into infected cells. These pathogen-associated molecular patterns (PAMPs) are recognized by specialized classes of innate immune receptors. By binding to PAMPs, these receptors induce powerful proinflammatory responses that neutralize infection and galvanize a robust adaptive immune response. One of the most important receptors for responding to viral pathogens is Retinoic Acid Inducible Gene 1 (RIG-I), which specifically recognizes double-stranded viral RNA and is required for defense against agents such as influenza, flaviviruses and hepaciviruses.

Extensive structural and biochemical studies of the RIG-I receptor suggest a diversity of potential strategies for modulating activity of the protein. There has been significant interest in the development of synthetic agonists for RIG-I, which show promise as antivirals, vaccine adjuvants, and antitumor agents, particularly for cancers that are refractory to checkpoint blockade therapy. However, just as controlled activation of the innate immune system is useful, controlled deactivation of innate receptors has therapeutic utility, and the design of RIG-I antagonists is of particular interest.

Hyperactivation of RIG-I and RLR-mediated signaling is associated with a number of severe autoimmune disorders and conditions that result from the inappropriate distribution or processing of host RNA molecules (a type of Damage Induced Molecular Pattern, or DAMP), which inappropriately activate RIG-I signaling and misdirect potent antiviral inflammatory responses. RIG-I activation plays a role in diseases that stem from malfunction of the human RNA decay machinery. This connection between such dysregulation and RIG-I activation suggests a role for RIG-I activity in common autoimmune disorders, such as type-I diabetes and Sjögren's syndrome.

In addition to diseases that result from inappropriate host RNA distribution, RIG-I hyperstimulation plays a role in other pathological conditions. For example, RIG-I is implicated in the catastrophic exacerbation of chronic obstructive pulmonary disease (COPD) that frequently accompanies viral infection. Further, there is evidence that induction of osteoarthritis involves the recruitment of fibroblast-like synoviocytes, and that inhibition of the RIG-I signaling pathway can dampen this response. More broadly, RIG-I provides an attractive target for the treatment of inflammatory disease, as activation of this protein leads to up-regulation of second-messengers that are currently targeted by standard therapies.

Despite its key role within the immune system, RIG-I remains a relatively unexplored small molecule drug target. Tools for the direct modulation of any innate immune receptor are rare, and given their potential utility for application in oncology, antimicrobial and autoimmune disease, innate immune modulators represent a major new frontier in chemical biology.

There still remains a need in the art for RIG-I antagonists. In certain embodiments, such compounds can be used for inhibiting RIG-I activity. In other embodiments, such compounds can be used for treating, ameliorating, and/or preventing a disease or disorder associated with defective distribution and processing of host RNA molecules. In yet other embodiments, such compounds can be used for treating, ameliorating, and/or preventing a disease or disorder associated with malfunction of the human RNA decay machinery. In yet other embodiments, such compounds can be used for treating, ameliorating, and/or preventing an autoimmune disorder, such as type-I diabetes and Sjögren's syndrome. In yet other embodiments, such compounds can be used for treating, ameliorating, and/or preventing COPD. In yet other embodiments, such compounds can be used for treating, ameliorating, and/or preventing an inflammatory disease. The present disclosure satisfies this need in the art.

### BRIEF DESCRIPTION OF THE DRAWINGS

The following detailed description of exemplary embodiments of the disclosure will be better understood when read in conjunction with the appended drawings. For the purpose of illustrating the disclosure, non-limiting embodiments are shown in the drawings. It should be understood, however, that the disclosure is not limited to the precise arrangements and instrumentalities of the embodiments shown in the drawings.

FIG. 1A illustrates the structure of RIG-I ΔCARDs bound to a 14 base pair RNA duplex with ADP▪BeF3 (PDBID:5E3H). FIG. 1B illustrates representative IC₅₀ curves for active compounds. Relative activity of a compound was calculated as a fraction of “no inhibitor” measurement and plotted vs. inhibitor concentration. Each data point represents a mean of n=3 determinations±the standard deviation. FIG. 1C illustrates a representative absorption titration spectra of RIG012 binding to RIG-I. Protein concentrations are indicated in figure legend. (Inset) Stoichiometry of RIG-I binding. Fraction bound (A) is plotted against fractional change in RIG-I concentration (ΔRIG-I). Slope=0.95±0.06. Data represent average of two independent experiments±SD.

FIG. 2 illustrates SAR examination for BVT compound (shown are IC₅₀ values for RIG-I ATPase). The three colored regions of the lead compound were explored for studying SAR. Functional region changes for optimization are highlighted in yellow.

FIGS. 3A-3C illustrate RIG-I inhibition by selected compounds in vivo. FIGS. 3A-3B: Inhibition of Luciferase activity in HEK293T cells cotransfected with reporters and treated with selected inhibitors as described elsewhere herein. Each bar represents the mean±SD of three experiments. IC₅₀ values were determined from corresponding dose-response curves (inset). FIG. 3C: Inhibition of RIG-I dependent IFNp and hRSAD2 expression in A549 cells in the presence of RIG012, monitored by qRT PCR (Methods). Samples were analyzed in triplicate and normalized to HPRT expression. Data represent average of two independent experiments±SD.

FIGS. 4A-4B illustrate q-RT-PCR controls. Gene expression levels are unaffected by amount of inhibitor used. FIG. 4A: Metabolic genes, FIG. 4B: Stress genes.

### DETAILED DESCRIPTION OF THE DISCLOSURE

The present disclosure provides compositions and methods for inhibiting RIG-I activity in a cell. In certain embodiments, the inhibitor is a RIG-I antagonist. In other embodiments, the compounds of the disclosure can be used for treating, ameliorating, and/or preventing a disease or disorder associated with defective distribution and processing of host RNA molecules. In yet other embodiments, the compounds of the disclosure can be used for treating, ameliorating, and/or preventing a disease or disorder associated with a malfunction of the human RNA decay machinery. In yet other embodiments, such compounds can be used for treating, ameliorating, and/or preventing an autoimmune disorder, such as type-I diabetes and Sjögren's syndrome. In yet other embodiments, such compounds can be used for treating, ameliorating, and/or preventing COPD. In yet other embodiments, such compounds can be used for treating, ameliorating, and/or preventing an inflammatory disease.

The RIG-I receptor plays a key role in the vertebrate innate immune system, where it functions as a sensor for detecting infection by RNA viruses. Although agonists of RIG-I show great potential as antitumor and antimicrobial therapies, antagonists of RIG-I remain undeveloped, despite the role of RIG-I hyperstimulation in a range of diseases, including COPD and autoimmune disorders. There is now a wealth of information on RIG-I structure, enzymatic function and signaling mechanism that can drive new drug design strategies. As shown elsewhere herein, enzymology, structural biology, and medicinal chemistry were used to identify and optimize a series of compounds that specifically modulate the innate immune signaling activity of RIG-I, with applications as tool compounds and therapeutics. In certain embodiments, the disclosure provides RIG-I antagonists that interact directly with the receptor, and which inhibit RIG-I signaling and interferon response in cells and animals.

The skilled artisan will understand that the disclosure is not limited to the exemplary therapies discussed herein. Further, the skilled artisan will understand that one or more therapies can be administered alone or in any combination. Still further, the skilled artisan will understand that one or more therapies can be administered in combination with any other type of therapy.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, selected methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±10%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, and so forth) from those organisms, tissues, cells or components thereof that display the “normal” (expected) respective characteristic. Characteristics that are normal or expected for one cell or tissue type might be abnormal for a different cell or tissue type.

A disease or disorder is “alleviated” if the severity of a symptom of the disease or disorder, the frequency with which such a symptom is experienced by a patient, or both, is reduced.

As used herein, the term “specifically bind” or “specifically binds,” as used herein, is meant that a first molecule (e.g., a target protein or a phosphatase) preferentially binds to a second molecule (e.g., a target protein ligand or a phosphatase ligand, respectively), but does not necessarily bind only to that second molecule. In certain embodiments, the binding is reversible. In other embodiments, the binding is irreversible (or non-reversible).

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount,” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation.

As used herein, the term “efficacy” refers to the maximal effect (Eₘₐₓ) achieved within an assay.

As used herein, an “instructional material” includes a publication, a recording, a diagram, or any other medium of expression which can be used to communicate the usefulness of a compound, composition, vector, or delivery system of the disclosure in the kit for effecting alleviation of the various diseases or disorders recited herein. Optionally, or alternately, the instructional material can describe one or more methods of alleviating the diseases or disorders in a cell or a tissue of a mammal. The instructional material of the kit of the disclosure can, for example, be affixed to a container which contains the identified compound, composition, vector, or delivery system of the disclosure or be shipped together with a container which contains the identified compound, composition, vector, or delivery system. Alternatively, the instructional material can be shipped separately from the container with the intention that the instructional material and the compound be used cooperatively by the recipient.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate).

Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, trifluoroacetic acid, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the disclosure include, for example, ammonium salts, metallic salts including alkali metal, alkaline earth metal and transition metal salts such as, for example, calcium, magnesium, potassium, sodium, and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine), and procaine. All of these salts may be prepared from the corresponding compound by reacting, for example, the appropriate acid or base with the compound.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure.

Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject, or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein and/or a symptom of a condition contemplated herein, with the purpose to cure, heal, alleviate, relieve, alter, remedy, ameliorate, improve or affect a condition contemplated herein and/or the symptoms of a condition contemplated herein. Such treatments may be specifically tailored or modified, based on knowledge obtained from the field of pharmacogenomics.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C≡N(—CN), —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

The term “alkylene” refers to a diradical of an alkyl group. Exemplary alkylene groups include —CH₂—, —CH₂CH₂—, and —CH₂C(H)(CH₃)CH₂—. The term “—(C₀ alkylene)-” refers to a bond. Accordingly, the term “—(C₀₋₃ alkylene)-” encompasses a bond (i.e., C₀) and a —(C₁₋₃ alkylene) group.

As used herein, the term “haloalkyl” means alkyl as defined above, substituted by one, two or three substituents selected from the group consisting of F, Cl, Br, and I.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized or substituted. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, —NH—(CH₂)ₘ—OH (m=1-6), —N(CH₃)—(CH₂)ₘ—OH (m=1-6), —NH—(CH₂)ₘ—OCH₃ (m=1-6), and —CH₂CH₂—S(═O)—CH₃. Up to two heteroatoms may be consecutive, such as, for example, —CH₂NH—OCH₃, or —CH₂CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “cycloalkyl” refers to a mono cyclic or polycyclic non-aromatic radical, wherein each of the atoms forming the ring (i.e. skeletal atoms) is a carbon atom. In certain embodiments, the cycloalkyl group is saturated or partially unsaturated. In other embodiments, the cycloalkyl group is fused with an aromatic ring. Cycloalkyl groups include groups having from 3 to 10 ring atoms. Illustrative examples of cycloalkyl groups include, but are not limited to, the following moieties:

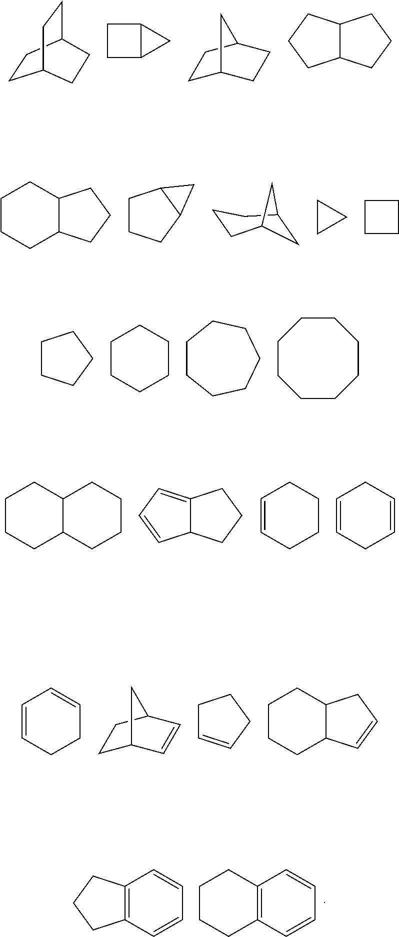

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.p

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl. Preferred examples are phenyl and naphthyl, most preferred is phenyl.

As used herein, the term “aryl-(C₁-C₃)alkyl” means a functional group wherein a one- to three-carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

The term “carbocyclyl” refers to a saturated or unsaturated carbocyclic ring system containing one or more rings (typically one, two or three rings). In certain embodiments, the carbocyclyl is a 3-12 membered carbocyclic ring, a 3-8 membered carbocyclic ring, or a 3-6 membered carbocyclic ring.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

The term “heteroalkylene” refers to an alkylene group in which one or more carbon atoms has been replaced by a heteroatom (e.g., N, O, or S). Exemplary heteroalkylene groups include —CH₂O—, —CH₂OCH₂—, and —CH₂CH₂O—. The heteroalkylene group may contain, for example, from 2-4, 2-6, or 2-8 atoms selected from the group consisting of carbon and a heteroatom (e.g., N, O, or S).

As used herein, the term “heterocycloalkyl” or “heterocyclyl” refers to a heteroalicyclic group containing one to four ring heteroatoms each selected from O, S and N. In certain embodiments, each heterocycloalkyl group has from 4 to 10 atoms in its ring system, with the proviso that the ring of said group does not contain two adjacent O or S atoms. In other embodiments, the heterocycloalkyl group is fused with an aromatic ring. In certain embodiments, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

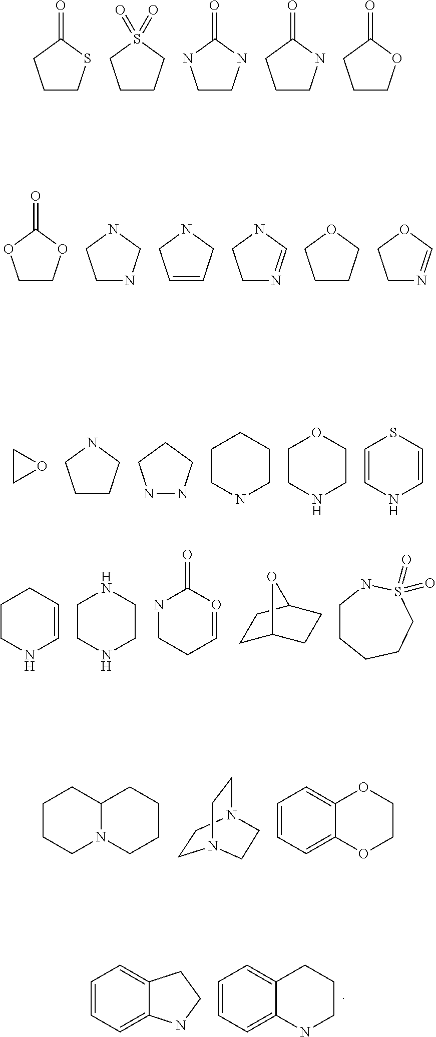

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “heteroaryl” or “heteroaromatic” refers to a heterocycle having aromatic character. A polycyclic heteroaryl may include one or more rings that are partially saturated. Examples include the following moieties.

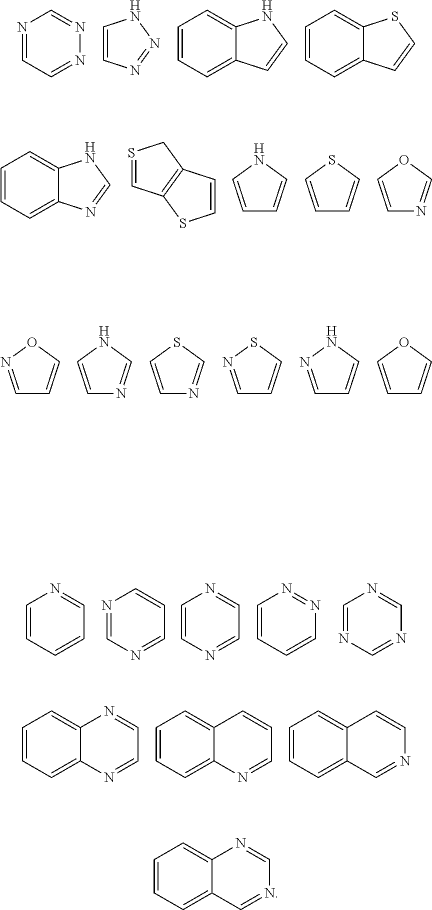

Examples of heteroaryl groups also include pyridyl, pyrazinyl, pyrimidinyl (particularly 2- and 4-pyrimidinyl), pyridazinyl, thienyl, pyrroyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

The term “heteroarylene” refers to a multi-valent (e.g., di-valent or trivalent) aromatic group that comprises at least one ring heteroatom. An exemplary “heteroarylene” is pyridinylene, which is a multi-valent radical of pyridine. For example, a divalent radical of pyridine is illustrated by the formula

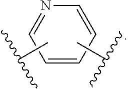

In certain embodiments, the “heteroarylene” is a divalent, 5-6 membered heteroaromatic group containing 1, 2, or 3 ring heteroatoms (e.g., O, N, or S).

The term “phenylene” refers to a multivalent radical (e.g., a divalent or trivalent radical) of benzene. To illustrate, a divalent radical of benzene is illustrated by the formula

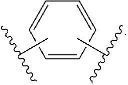

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two.

As used herein, the term “optionally substituted” means that the referenced group may be substituted or unsubstituted. In certain embodiments, the referenced group is optionally substituted with zero substituents, i.e., the referenced group is unsubstituted. In other embodiments, the referenced group is optionally substituted with one or more additional group(s) individually and independently selected from groups described herein.

In certain embodiments, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂ alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In other embodiments, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet other embodiments, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

In certain embodiments, an optional substituent is selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, an optional substituent is selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —OR^{b}, and —C(═O)N(R^{b})(R^{b}), wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, an optional substituent is selected from the group consisting of C₁-C₆ alkyl, —OH, C₁-C₃ haloalkyl, C₁-C₆ alkoxy, C₃-C₈ cycloalkyl, C₃-C₈ cycloalkoxy, halo, and —CN.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

The disclosure provides a compound of formula (I), or a salt, solvate, isotopically labelled derivative, stereoisomer, tautomer, or geometric isomer thereof:

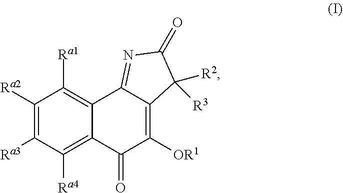

wherein:

R¹ is selected from the group consisting of H, optionally substituted C₁-C₆ alkyl, optionally substituted C₂-C₆ alkenyl, optionally substituted C₂-C₆ alkynyl, optionally substituted C₃-C₈ cycloalkyl, optionally substituted —(C₀-C₆ alkylene)-phenyl, optionally substituted —(C₀-C₆ alkylene)-naphthyl, and optionally —(C₀-C₆ alkylene)-substituted heteroaryl;

R² is selected from the group consisting of H, optionally substituted C₁-C₆ alkyl, optionally substituted C₂-C₆ alkenyl, optionally substituted C₂-C₆ alkynyl, and optionally substituted C₃-C₈ cycloalkyl;

R³ is selected from the group consisting of H, optionally substituted C₁-C₆ alkyl, optionally substituted C₂-C₆ alkenyl, optionally substituted C₂-C₆ alkynyl, and optionally substituted C₃-C₈ cycloalkyl;
or R² and R³ combine to form optionally substituted C₂-C₇ alkylene;

each occurrence of R^{a1}, R^{a2}, R^{a3}, and R^{a4} is independently selected from the group consisting of H, F, Cl, Br, I, CN, C₁-C₆ alkyl, C₃-C₈ cycloalkyl, phenyl, C₁-C₆ hydroxyalkyl, C₁-C₆ haloalkyl, C₁-C₆ haloalkoxy, (C₁-C₆ alkoxy)-C₀-C₆ alkylene, —NR^{b}R^{b}, —OR^{b}, —C(═O)OR^{b}, and —C(═O)N(R^{b})(R_{b}),
wherein each occurrence of R^{b} is independently H, C₁-C₆ alkyl, or C₃-C₈ cycloalkyl.

In certain embodiments, R¹ is H. In certain embodiments, R¹ is methyl. In certain embodiments, R¹ is ethyl. In certain embodiments, R¹ is isobutyl. In certain embodiments, R¹ is benzyl. In certain embodiments, R¹ is —CH₂-naphthyl. In certain embodiments, R¹ is —CH₂—(2-benzimidazolyl). In certain embodiments, R¹ is optionally substituted methyl. In certain embodiments, R¹ is optionally substituted ethyl. In certain embodiments, R¹ is optionally substituted isobutyl. In certain embodiments, R¹ is optionally substituted benzyl. In certain embodiments, R¹ is optionally substituted —CH₂-naphthyl. In certain embodiments, R¹ is optionally substituted —CH₂—(2-benzimidazolyl).

In certain embodiments, R² is H. In certain embodiments, R² is methyl. In certain embodiments, R² is ethyl. In certain embodiments, R² is optionally substituted methyl. In certain embodiments, R² is optionally substituted ethyl.

In certain embodiments, R³ is H. In certain embodiments, R³ is methyl. In certain embodiments, R³ is ethyl. In certain embodiments, R³ is optionally substituted methyl. In certain embodiments, R³ is optionally substituted ethyl.

In certain embodiments, R² and R³ combine to form —CH₂—. In certain embodiments, R² and R³ combine to form —(CH₂)₂—. In certain embodiments, R² and R³ combine to form —(CH₂)₃—. In certain embodiments, R² and R³ combine to form —(CH₂)₄—. In certain embodiments, R² and R³ combine to form —(CH₂)₅—. In certain embodiments, R² and R³ combine to form —(CH₂)₆—. In certain embodiments, R² and R³ combine to form —(CH₂)₇—.

In certain embodiments, the compound of formula (I) is selected from the group consisting of:
4-Hydroxy-3,3-dimethyl-2H,3H,5H-benzo[g]indole-2,5-dione

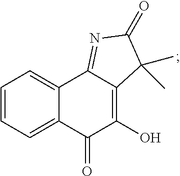 [SMILES: CC1(C)C(=O)N=C2C1=C(O)C(=O)c1ccccc12]

4-Methoxy-3,3-dimethyl-2H,3H,5H-benzo[g]indole-2,5-dione

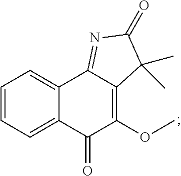 [SMILES: COC1=C2C(=NC(=O)C2(C)C)c2ccccc2C1=O]

4-Benzyloxy-3,3-dimethyl-2H,3H,5H-benzo[g]indole-2,5-dione

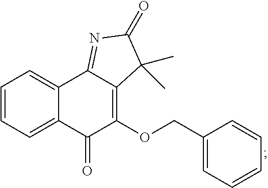 [SMILES: CC1(C)C(=O)N=C2C1=C(OCc1ccccc1)C(=O)c1ccccc12]

4-(2,2-Dimethylethoxy)-3,3-dimethyl-2H,3H,5H-benzo[g]indole-2,5-dione

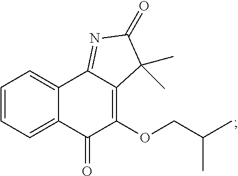 [SMILES: CC(C)COC1=C2C(=NC(=O)C2(C)C)c2ccccc2C1=O]

4-Ethoxy-3,3-dimethyl-2H,3H,5H-benzo[g]indole-2,5-dione

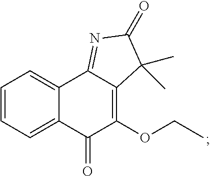 [SMILES: CCOC1=C2C(=NC(=O)C2(C)C)c2ccccc2C1=O]

4-(1H-1,3-Benzodiazol-2-ylmethoxy)-3,3-dimethyl-2H,3H,5H-benzo[g]indole-2,5-dione

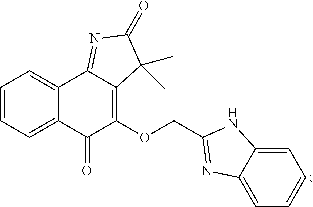 [SMILES: CC1(C)C(=O)N=C2C1=C(OCc1nc3ccccc3[nH]1)C(=O)c1ccccc12]

4-Hydroxy-2,5-dihydrospiro[benzo[g]indole-3,1′-cyclohexane]-2,5-dione

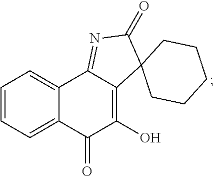 [SMILES: O=C1C(O)=C2C(=NC(=O)C23CCCCC3)c2ccccc21]

4-(Benzyloxy)-2,5-dihydrospiro[benzo[g]indole-3,1′-cyclohexane]-2,5-dione

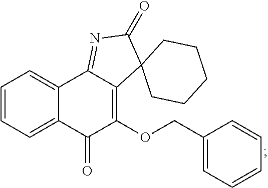 [SMILES: O=C1C(OCc2ccccc2)=C2C(=NC(=O)C23CCCCC3)c2ccccc21]

3,3-Diethyl-4-hydroxy-2H,3H,5H-benzo[g]indole-2,5-dione

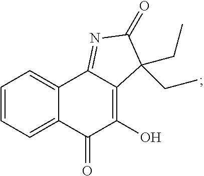 [SMILES: CCC1(CC)C(=O)N=C2C1=C(O)C(=O)c1ccccc12]

4-Benzyloxy-3,3-diethyl-2H,3H,5H-benzo[g]indole-2,5-dione

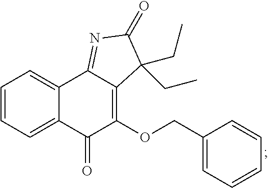 [SMILES: CCC1(CC)C(=O)N=C2C1=C(OCc1ccccc1)C(=O)c1ccccc12]

and
3,3-Dimethyl-4-(naphthalen-2-ylmethoxy)-2H,3H,5H-benzo[g]indole-2,5-dione

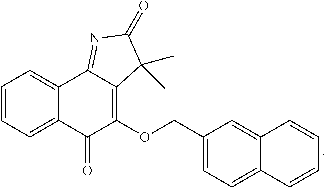 [SMILES: CC1(C)C(=O)N=C2C1=C(OCc1ccc3ccccc3c1)C(=O)c1ccccc12]

Compounds of the disclosure can be prepared by the general schemes and/or procedures described herein, using the synthetic method known by those skilled in the art.

The compounds of the disclosure may possess one or more stereocenters, and each stereocenter may exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the disclosure may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the disclosure are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O ¹⁸O, ³²P, and ³⁵S.

In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

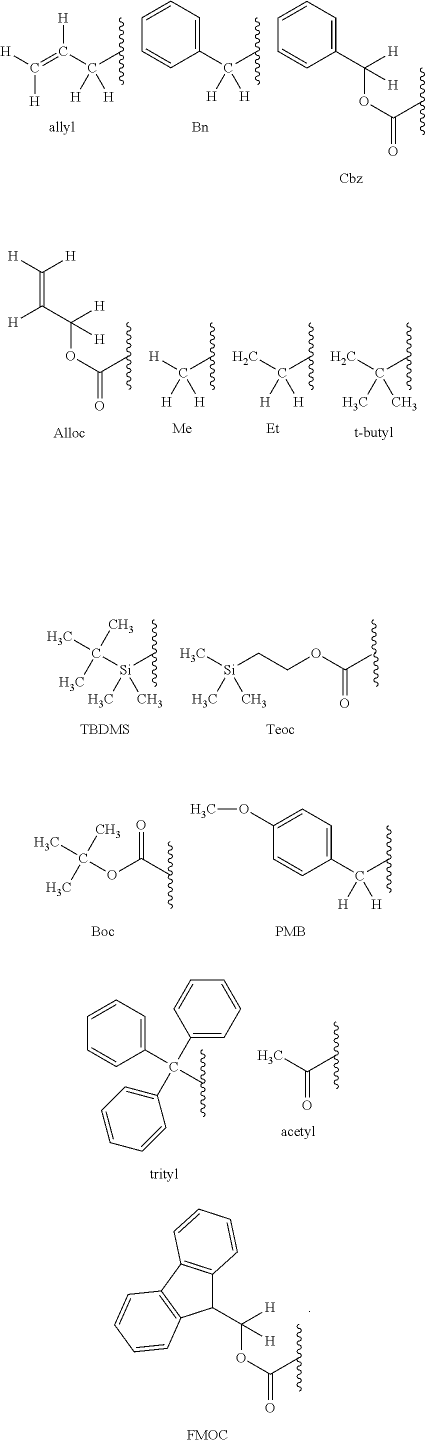

Other protecting groups, plus a detailed description of techniques applicable to the creation of protecting groups and their removal are described in Greene & Wuts, Protective Groups in Organic Synthesis, 3^{rd} Ed., John Wiley & Sons, New York, N.Y., 1999, and Kocienski, Protective Groups, Thieme Verlag, New York, N.Y., 1994, which are incorporated herein by reference for such disclosure.

### Compositions

The disclosure includes a pharmaceutical composition comprising at least one compound of the disclosure and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

### Methods

The disclosure provides compositions and methods for inhibiting RIG-I activity. The disclosure further provides compositions and methods for treating, ameliorating, and/or preventing a disease or disorder associated with defective distribution and processing of host RNA molecules. The disclosure further provides compositions and methods for treating, ameliorating, and/or preventing a disease or disorder associated with malfunction of the human RNA decay machinery. The disclosure further provides compositions and methods for treating, ameliorating, and/or preventing an autoimmune disorder, such as type-I diabetes and Sjögren's syndrome. The disclosure further provides compositions and methods for treating, ameliorating, and/or preventing COPD. The disclosure further provides compositions and methods for treating, ameliorating, and/or preventing an inflammatory disease.

In certain embodiments, the method comprises administering to the subject a therapeutically effective amount of a compound of the disclosure.

Vertebrate animals include, but are not limited to, fish, amphibians, birds, and mammals. Mammals include, but are not limited to, rats, mice, cats, dogs, horses, sheep, cattle, cows, pigs, rabbits, non-human primates, and humans. In a specific embodiment, the mammal is human.

### Administration/Dosing

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this disclosure may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle.

The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

Compounds of the disclosure for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the disclosure) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound or conjugate of the disclosure, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

### Pharmaceutical Compositions

The present disclosure provides a pharmaceutical composition comprising at least one nucleic acid molecule of the present disclosure and a pharmaceutically acceptable carrier. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents. Other active agents useful in the present disclosure include anti-inflammatories, including corticosteroids, and immunosuppressants, chemotherapeutic agents, antibiotics, antivirals, antifungals, and the like.

Controlled- or sustained-release formulations of a pharmaceutical composition of the disclosure may be made using conventional technology, using for example proteins equipped with pH sensitive domains or protease-cleavable fragments. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, micro-particles, liposomes, or microspheres or a combination thereof to provide the desired release profile in varying proportions. Suitable controlled-release formulations known to those of ordinary skill in the art, including those described herein, can be readily selected for use with the pharmaceutical compositions of the disclosure. Thus, single unit dosage forms suitable for oral administration, such as tablets, capsules, gel-caps, and caplets, which are adapted for controlled-release are encompassed by the present disclosure.

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the disclosure may be administered in the form of microparticles, for example, by injection or in the form of wafers or discs by implantation.

In a preferred embodiment of the disclosure, the compounds of the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences (1985, Genaro, ed., Mack Publishing Co., Easton, Pa.), which is incorporated herein by reference.

Routes of administration of any of the compositions of the disclosure include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. The formulations and compositions that would be useful in the present disclosure are not limited to the particular formulations and compositions that are described herein.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

### Kits

The disclosure also provides kits including a compound and/or a composition of the disclosure, and optionally another therapeutic agent, as described herein elsewhere, and instructions for its use. The instructions will generally include information about the use of the compositions in the kit for the treating, ameliorating, and/or preventing the diseases and disorders contemplated here. The instructions may be printed directly on a container inside the kit (when present), or as a label applied to the container, or as a separate sheet, pamphlet, card, or folder supplied in or with the container.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction and/or treatment conditions, with art-recognized alternatives and using no more than routine experimentation, are within the scope of the present application.

### EXPERIMENTAL EXAMPLES

The disclosure is further described in detail by reference to the following experimental examples. These examples are provided for purposes of illustration only, and are not intended to be limiting unless so specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

### Materials and Methods

### Protein Expression

Wild-type hs RIG-I was cloned into the pET-SUMO vector (Life Technologies) using the manufacturer's protocols.

The RIG-I expression plasmid was transformed into Rosetta II(DE3) Escherichia coli cells (Novagen) using 150 ng/25 μL commercial cell stocks and grown in LB media supplemented with 50 mM Potassium Phosphate pH 7.4 and 1% glycerol. Expression was induced by the addition of isopropyl-β-D-thiogalactopyranoside (IPTG) to a final concentration of 0.5 mM. Cells were grown for 24 h at 16° C., then harvested by centrifugation, resuspended in lysis buffer (20 mM Phosphate pH 7.4, 500 mM NaCl, 10% glycerol, 5 mM β-mercaptoethanol (PME)) to a final volume of 50 ml and frozen at −80° C. For lysis, frozen pellets were thawed at room temperature, then resuspended in an additional 200 ml lysis buffer per 4 L pellet. Cells were lysed by passage through a microfluidizer at 15,000 psi, and the lysate was clarified by ultracentrifugation at 100,000×g for 30 min. Soluble lysate was incubated on 2.5 ml Ni-NTA beads (Qiagen), washed with lysis buffer containing an additional 40 mM imidazole, then eluted in Ni elution buffer (25 mM HEPES pH 8.0, 150 mM NaCl, 220 mM Imidazole, 10% glycerol, 5 mM PME). Eluted protein was bound to a HiTrap Heparin HP column (GE Biosciences), washed in buffer containing 150 mM NaCl and eluted stepwise at 0.65 M NaCl. The SUMO tag was then removed by incubation with SUMO protease for 2 h at 4° C. Finally, monomeric protein was collected by passage over a HiPrep 16/60 Superdex 200 column (GE Biosciences) in gel filtration buffer (25 mM MOPS pH 7.4, 300 mM NaCl, 5% glycerol, 5 mM PME). Peak fractions were concentrated to 10-20 μM using a centrifugal concentrator with a 50 kD molecular weight cutoff (Millipore).

The native, full-length protein was expressed and purified using methods adapted from published protocols. Concentrations were determined spectrophotometrically using an extinction coefficient of ϵ=99,700 M⁻¹ cm⁻¹ at λ=280 nm. Protein preparations were separated into aliquots, flash frozen using liquid nitrogen and stored at −80° C.

### ADP-Glo ATPase Assay

For the ADP-Glo assay system (V9101, Promega), RIG-I protein was diluted to 10 nM in assay buffer (25 mM MOPS pH 7.4, 150 mM NaCl, 2 mM DTT, and 2 mM Mg²). For HTS experiments, Poly I:C RNA was used for RIG-I activation, and added to a final concentration of 0.4 μg/ml. To initiate the ATPase reactions, ATP was added to a final concentration of 200 μM. To test compounds, a 12-point serial dilution of the small molecule of interest was combined with DMSO, keeping the DMSO levels at a constant 1% throughout the entire drug dilution. Final drug concentrations ranged from 0 to 200 μM. The final volume for reaction was 20 μL per well, measured in a Corning 384-well plate (Plate ID: 3658). Reactions were allowed to proceed at room temperature for one hour, after which 2.5 μL of the ADP-Glo Reagent was added to each well. Samples were incubated at 25° C. for 45 min to terminate the ATPase reaction and deplete the remaining ATP. Next, 5 μL of Kinase Detection Reagent was added to each well in order to convert the ADP product back to ATP, which is then used as a substrate for the luminescent reporter, which was read on a Biotek Synergy HI Plate Reader.

Slopes from the twelve point curves were fit to a non-competitive, four variable inhibition curve in order to obtain IC₅₀ values:

$$Y = Bottom + \frac{( Top - Bottom )}{( 1 + 10 * Log ( IC 50 ) - X ) * Hillslope} \qquad ( 1 )$$

where X is Log₁₀ [compound], Y is luminescence, Top and Bottom are the plateaus at Yₘᵢₙ and Yₘₐₓ. The Log₁₀ IC₅₀ is solved in the same units as X. HillSlope is a unit-less factor designed to account for complex variables in the standard relationship between compound binding and Y value response. Each point was a based on two trials of three biological replicates with error bars representing the Population Standard Deviation across all values.

### High Throughput Screening and Synthetic Compounds:

The commercially available ADP-Glo luminescent reporter assay (Promega) was adapted for the RIG-I ATPase system and optimized to achieve a reproducible Z′ score between 0.7 and 0.9, and tested for compatibility with up to 2% DMSO vehicle. Using this assay, a library of over 11,000 compounds spanning 11 libraries, including the ChemBridge, Enzo Kinase Inhibitor, Enzo Phosphatase Inhibitor, ChemDiv and Nuclear Receptor Libraries, was screened. The threshold for hit selection was set at 20% inhibition compared to vehicle control, yielding 33 hits or a hit rate of 0.3%. After HTS, all compounds created for SAR and optimization study were synthesized as described elsewhere herein.

### NADH-Coupled ATPase Assay

For the NADH-coupled assay, RIG-I protein was diluted into ATPase assay buffer (25 mM MOPS pH 7.4, 150 mM KCl, 2 mM DTT) to a final concentration of 10 nM for early compounds and then 20 nM for visualizing more potent inhibitors. In this case, RIG-I was activated by a well-defined synthetic RNA hairpin duplex (SLR10-OH), which was added to a final concentration of 250 nM. A coupled assay mixture consisting of 1 mM NADH, 100 U/ml lactic dehydrogenase, 500 U/ml pyruvate kinase, 2.5 mM phosphoenol pyruvic acid was added to the sample. A 12-point serial dilution of the compound of interest was added in a DMSO vehicle such that DMSO concentrations were kept at a constant 2% over the entire drug dilution. The final concentration ranges for drug dilutions varied from compound to compound based on the potency of the small molecule in question. The dilutions covered ranges from 0 to as high as 100 μM (see association figures). Samples were incubated for at least 1 hour at RT. Reactions were initiated by the addition of a 1:1 ATP/MgCl₂ mix to a final concentration of 5 mM.

The rate of ATP hydrolysis was determined indirectly by monitoring the conversion of NADH to NAD⁺ that results in a loss of sample absorbance at 340 nM. Absorbance was measured over a 20 min time course using a sweeping read at 10 second intervals and a gain setting of 120 on a Biotek Synergy H1 Plate Reader. Mean k_{obs} values were extrapolated for each time course and plotted against drug concentration, then fit to Eq. (1) to determine IC₅₀ values. Each point was a based on two trials of three biological replicates with error bars representing the Standard deviation.

### Cell-Based Assays for RIG-I Inhibition and Compound Toxicity

Two different assays were used for evaluating RIG-I inhibition in cells. In the first assay, HEK 293T cells were used to test the influence of compounds on RIG-I signaling activity while simultaneously reporting on relative toxicity. In this case, inhibitor compounds were introduced via growth media replacement rather than by direct addition to cell plates. Specifically, HEK 293T cells (which express RIG-I only under the control of a transfected plasmid) were grown and maintained in T75 flasks containing Dulbecco's Modified Eagle Medium, DMEM (Life Technologies) supplemented with 10% heat-inactivated fetal bovine serum (Hyclone) and non-essential amino acids (Life Technologies). For the IFN-β induction assays, 0.5 ml of cells at 65,000 cells/ml was placed (seeded) in 24-well plates. After 24 h, each well of cells was transfected with 5 ng WT pUNO-hRIG-I (Invivogen), 6 ng pRL-TK constitutive Renilla luciferase reporter plasmid (Promega) and 150 ng of an IFN-β/Firefly luciferase reporter plasmid using the Lipofectamine 2000 transfection reagent (Life Technologies) following the manufacturer's protocol. Protein expression was allowed to proceed for 24 hours. In parallel, a six-point dilution series of each compound of interest was made in a DMSO vehicle and then diluted further into DMEM such that a constant DMSO concentration of 1% is maintained over the entire drug dilution. The DMEM growth media was aspirated from each well of cells and replaced with DMEM that contains the drug. After 30 minutes, the cells were challenged by transfection with 1 μg of the synthetic stem-loop RNA (SLR14) in Lipofectamine 2000. After 5-6 hours, cells were harvested for luminescence analysis in the following manner: Growth media was aspirated from each well, and 100 μl of passive lysis buffer (Promega) was added. Lysis proceeded for 15 min at room temperature. Lysates were clarified by centrifugation, and 20 μl samples of the supernatant were transferred to a 96-well assay plate for analysis using the Dual-Luciferase Reporter Assay System (Promega). Luminescence was measured using a Biotek Synergy H1 plate reader. The resulting Firefly luciferase activity (i.e. the induction of IFN-0) was normalized to the activity of the constitutively expressed Renilla luciferase to account for differences in confluency, transfection efficiency and cell viability across sample wells.

In the second assay, inhibition of RIG-I that is endogenously expressed in A549 cells was evaluated by monitoring IFN and ISG levels by qRT-PCR. A549 cells were propagated in T75 flasks containing Dulbecco's Modified Eagle Medium, DMEM, (Life Technologies) supplemented with 10% heat-inactivated fetal bovine serum (Hyclone) and Non-Essential Amino Acids (Life Technologies). For the assay, 0.5 ml of cells at 100,000 cells/ml was seeded in 24-well plates. After 24 h, DMEM growth media was replaced with media containing drug, as described above for the IFN induction assay using HEK293T cells. After 30 minutes, the cells were transfected with 1 μg of the synthetic stem-loop RNA (SLR14), using the Lipofectamine 2000 transfection reagent. After 5-6 hours, cells were harvested and total RNA was isolated using E.Z.N.A total RNA isolation kit (Omega Bio-tek), following the manufacturer's instructions. Genomic DNA contamination was removed by DNase I treatment (Omega Bio-tek) directly on the Mini column. Total RNA was reverse-transcribed into cDNA using iScript Reverse Transcription Supermix (Bio-RAD) according to the manufacturer's instructions. The qRT-PCR was performed using iTaq Universal SYBR Green Supermix and a CFX384 Touch Real-Time PCR Detection System (Bio-Rad). Each sample was analyzed in triplicate and normalized to HPRT expression. Gene expression quantification was performed according to the Livak ΔΔCt method.

The following primer sets were used to amplify indicated targets:

| | HPRT: |
|---|---|
| (SEQ ID NO: 1) | |
| | TGGTCAGGCAGTATAATCCAAAG |
| | and |
| (SEQ ID NO: 2) | |
| | TTTCAAATCCAACAAAGTCTGGC |
| | GAPDH: |
| (SEQ ID NO: 3) | |
| | GCAAGAGCACAAGAGGAAGA |
| | and |
| (SEQ ID NO: 4) | |
| | CTACATGGCAACTGTGAGGA |
| | ACTB: |
| (SEQ ID NO: 5) | |
| | TTCCAGCAGATGTGGATCAG |
| | and |
| (SEQ ID NO: 6) | |
| | GGTGTAACGCAACTAAGTCA |
| | p21: |
| (SEQ ID NO: 7) | |
| | TGCCCAAGCTCTACCTTC |
| | and |
| (SEQ ID NO: 8) | |
| | GACAGTGACAGGTCCACAT |
| | BAX: |
| (SEQ ID NO: 9) | |
| | CACCAGCTCTGAGCAGATC |
| | and |
| (SEQ ID NO: 10) | |
| | GCTGCCACTCGGAAAAAG |
| | hRSAD2: |
| (SEQ ID NO: 11) | |
| | TCGCTATCTCCTGTGACAGC |
| | and |
| (SEQ ID NO: 12) | |
| | CACCACCTCCTCAGCTTTTG |

### Example 1: Identification of RIG-I Antagonists

In order to identify small molecules capable of inhibiting RIG-I activity, a high throughput small molecule screening strategy was designed and optimized, based on the ADP-Glo luminescent reporter system, which was selected due to its high sensitivity and stability. The assay was optimized to achieve a reproducible Z′ score (19551358) between 0.7 and 0.9, and tested for compatibility with up to 2% DMSO vehicle. From an initial pool of 11,000 compounds, 15 were selected for validation and dose-response analysis based on molecular weight, available functional moieties, and predicted solubility coefficient.

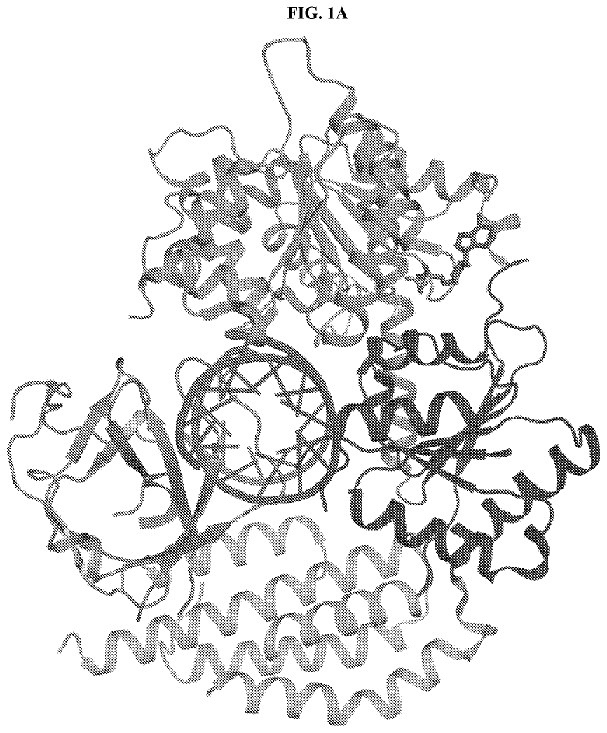

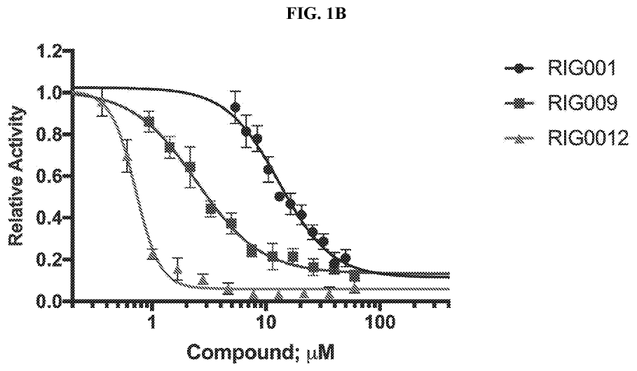

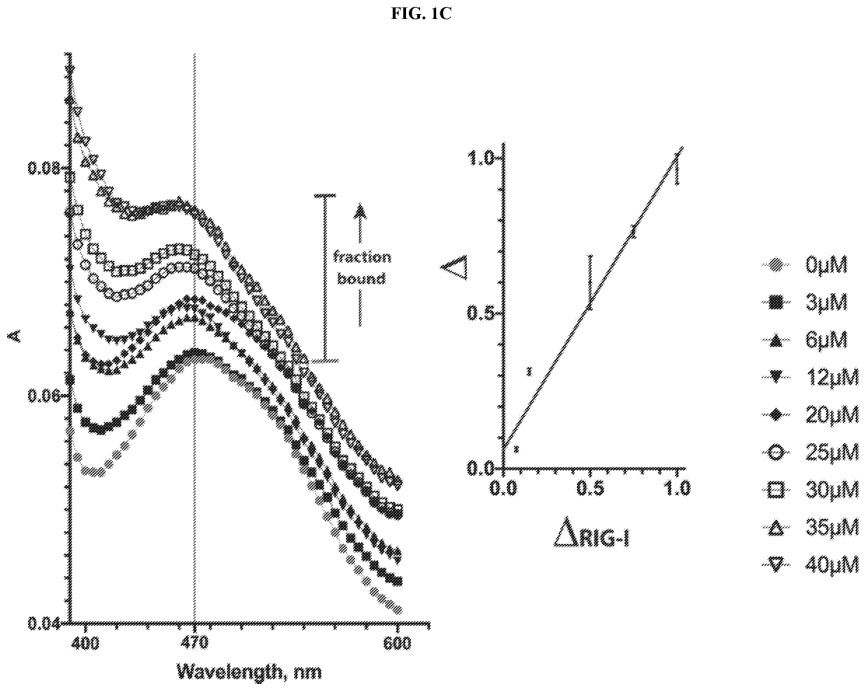

Of these 15 compounds, 9 exhibited dose-dependent ATPase inhibition with IC₅₀ values ranging from low micromolar to millimolar. The best hit from the active compound set was 4-hydroxy-3,3-dimethyl-2H-benzo[g]indole-2,5(3H)-dione, which was previously annotated as BVT.948 (henceforth designated RIG-001, Table 1). This compound displayed an apparent IC₅₀ value of 19±3 μM, and was therefore selected as a candidate for further optimization. In addition to the primary ADP-GLO assay, activity of all compound variants was quantitated using an orthogonal, NADH-coupled ATPase assay that was previously optimized for studies of RIG-I enzymology. This approach eliminated the possibility of spectroscopic interference caused by by-products of ATP hydrolysis in the ADP-Glo assay, and resulted in more accurate IC₅₀ values. Results from the two assays are in good agreement, as indicated by the fact that RIG001 has an IC₅₀ value of 12±1 M in the coupled assay system (Table 1, FIG. 1).

### Example 2: Structure-Activity Relationships and Optimization of RIG-001 Derivatives

Having established that RIG-001 is a bonafide inhibitor of RIG-I, the relative importance of compound functional groups was assessed and optimized using iterative medicinal chemistry (FIG. 2, Table 1). Three derivatives of the lead compound RIG-001 were synthesized with methylations at potentially important functional moieties (RIG002, RIG003 and RIG004, Table 1 and Methods). Whereas methylation of the imino (R⁴) or keto (R⁵) moieties of the indole eliminated inhibitory activity, methylation of the indole hydroxyl group (R¹) resulted in slightly greater potency than the original RIG001 (RIG002, Table 1, FIG. 2).

The adjacent keto and hydroxyl moieties of RIG001 suggested that compounds in this lead series might form undesirable covalent adducts. Without wishing to be limited by any theory, the fact that large lipophilic substituents at C2 and on the C3 oxygen enhance IC₅₀ values suggests that this region is chemically unreactive. The observed trend implies that the compounds have improved interactions with a nearby lipophilic pocket. By contrast, modifications to the indole ketone (R⁵) resulted in a loss of function (as in RIG004), suggesting that the ketone is an essential component of the minimal pharmacophore.

The observation that methyl substitution at R⁴ (RIG002) improves potency suggests that this position might tolerate substituents that enhance compound efficacy. To evaluate this, a series of derivatives (RIG005-RIG008) was created in which various hydrophobic moieties were appended to the hydroxyl at R¹ (Table 1). Indeed, IC₅₀ values of these compounds improved as larger alkyl and aryl groups are introduced at R¹, consistent with results from RIG002 (Table 1).

Having established that hydrophobic substitutions at R¹ enhanced compound potency, the effects of substitutions at the R²/R³ positions were next evaluated. A second set of derivatives annotated RIG009-RIG013 were synthesized in which modifications at R²/R³ were combined with substitutions at R¹. In compounds RIG009 and RIG0010, the methyl groups at R²/R³ were substituted with a fused cyclohexane ring in the context of either a hydroxyl or methylbenzene moiety at position R¹ (RIG009 and RIG010 respectively, Table 1). Compounds RIG011 and RIG012 have more conservative ethyl substitutions at R²/R³, also in the context of an R¹ hydroxyl and methylbenzene respectively. Compound RIG009 has an IC₅₀ value of 2.4±0.3 μM (Table 1), indicating that the fused R²/R³ site was still functional and suggesting that it may facilitate protein binding. In contrast, compound RIG010 showed a decrease in potency relative to RIG005, which contains the methylbenzene substitution at R¹, and RIG009, which contains the cyclohexane substitution at R²/R³. This suggests that the addition of several bulky groups in this region of the molecule is sterically problematic, as shown by the higher IC₅₀ value obtained for RIG010. Compounds RIG011 and RIG012 both exhibited nanomolar IC₅₀ values, indicating that ethyl groups at R²/R³ substantially increase compound efficacy in the presence of either a hydroxyl (RIG011) or methylbenzene (RIG012) at R1.

Based on the above results, a compound (RIG013) was synthesized in an effort to further explore the steric limitations of the lead series. This compound exhibited a higher IC₅₀ value (Table 1) than a similar compound bearing a methylbenzene substitution at R¹ and it was therefore determined that the methylbenzene moiety (on RIG012) was a favorable size for substituents at the R¹ site.

Although IC₅₀ values provide a valuable metric of association for enzyme inhibitors, it was of interest to determine directly whether the lead compound, RIG012, binds to the RIG-I protein. Binding studies were facilitated by the fact that RIG012 contains a strong chromophore that absorbs in the visible range (λₘₐₓ of 470 nm), making it possible to conduct classical absorbance titrations of the compound with increasing amounts of RIG-I. Upon binding to RIG-I, the 470 nm absorbance band of RIG012 undergoes a dramatic hyperchromic shift to higher intensity values, and the magnitude of this shift is proportional to the relative fraction of bound small molecule (FIG. 1C). Because the concentrations of both RIG012 and protein are above the IC₅₀ value (Table1), the titration data were obtained in the stoichiometric binding regime (FIG. 1C). For each fractional change in RIG-I concentration, there was an equivalent change in the fraction of RIG012 bound, indicating formation of 1:1 complex.

### Example 3: Inhibition of RIG-I Signaling in Cells

Having established that the compounds directly interact with the RIG-I protein and modulate its function, influence of the compounds on RIG-I signaling was examined in two different cell lines used for monitoring interferon induction and gene expression: HEK293T embryonic kidney cells and A549 lung epithelial cells. The HEK293T cell line can be used as a reporter system for RIG-I activation as this cell line lacks endogenous RIG-I expression and is therefore dependent on transfection of exogenous expression plasmids. In certain embodiments, compound efficacy as evaluated in this system reports mainly on RIG-I activity. The A549 cell line is successfully used as a model system for endogenous RIG-I activation.

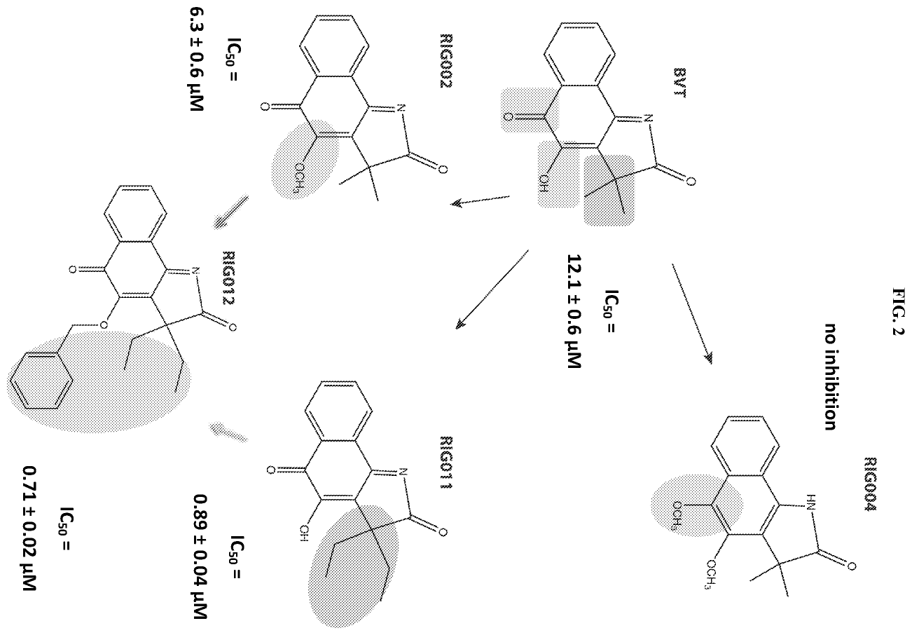

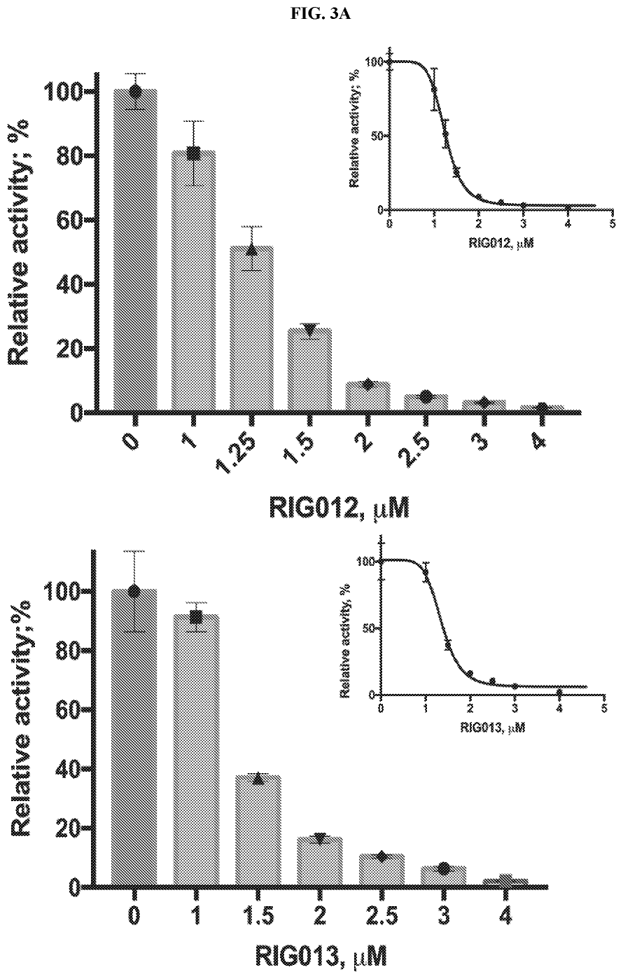

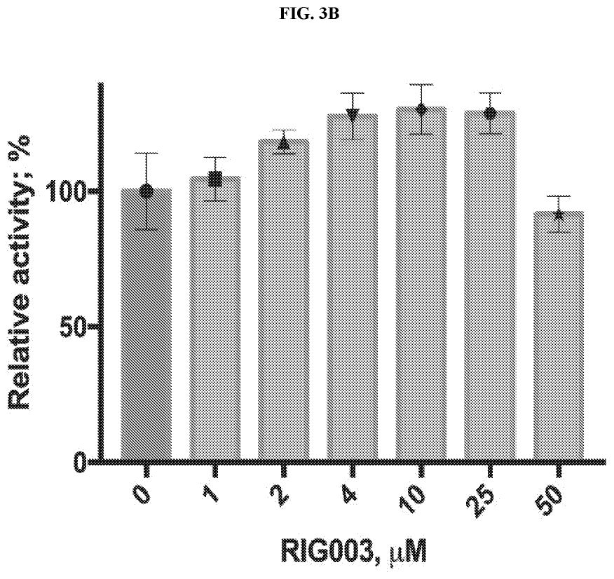

The experiments in HEK293T cells utilize a dual-luciferase system that reports on levels of RIG-I stimulated IFN induction upon treatment with inhibitors, while an internal control reporter simultaneously provides a metric of cell viability. HEK293T cells lack endogenous RIG-I expression and they are transfected with plasmids encoding RIG-I (or mutants thereof) in order to evaluate receptor-specific signaling. Upon introduction of compound into the growth medium, potent, dose-dependent inhibition of RIG-I signaling was observed upon treatment with RIG012 (FIG. 3A). Fitting the data to dose-response curve, an IC₅₀ value of 1.25 μM was determined for the compound in-vivo (FIG. 3A inset). The very closely-related inhibitor RIG013 showed IC₅₀ of 1.36 μM (FIG. 3A bottom panel inset), in agreement with biochemically-determined values for RIG-I inhibition. By contrast, inactive compounds did not influence levels of IFN induction or RIG-I response (see RIG003, FIG. 3B).

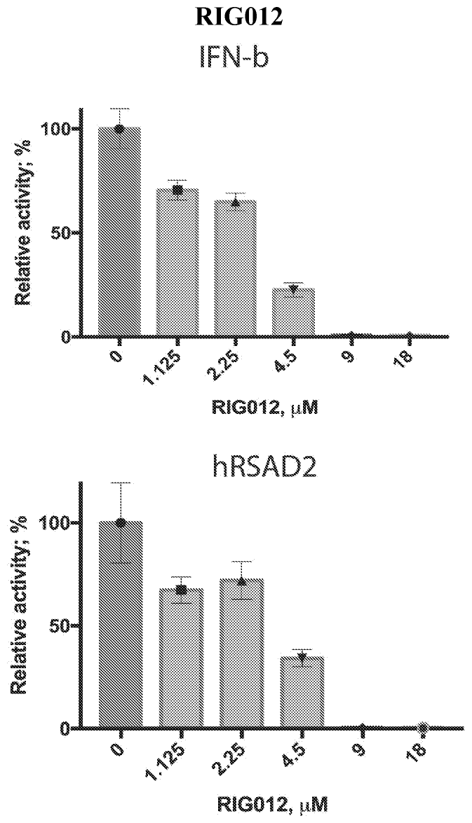

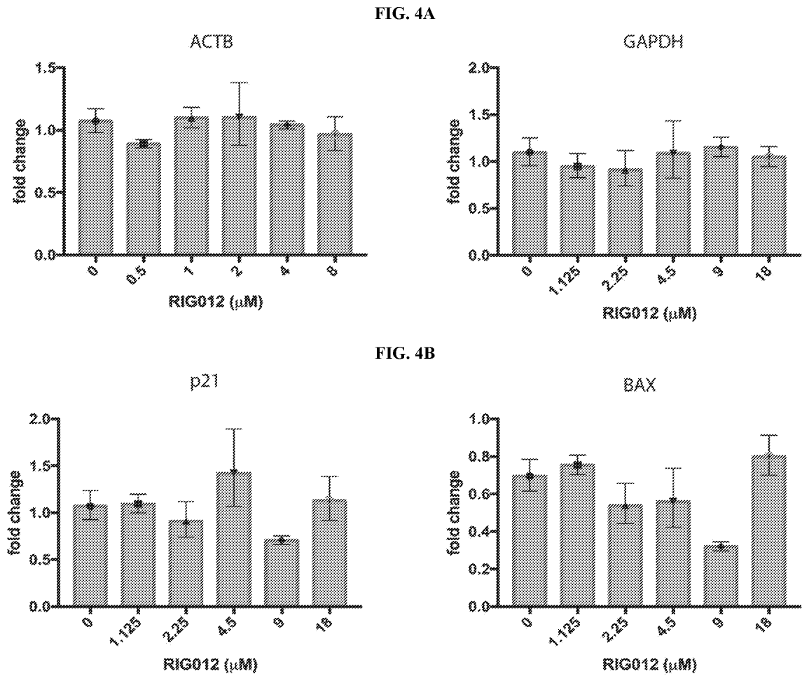

These experiments were consistent with a target-specific response to the compounds. Further, it was determined whether the compounds inhibit signaling in cells where RIG-I is endogenously expressed. To that end, A549 human lung epithelial cells, which exhibit constitutive RIG-I expression, were employed; qRT-PCR was used to monitor RIG-I-dependent expression of IFN and interferon-stimulated genes (ISGs, such as hRsad2 (viperin), FIG. 3C). To evaluate interference with cell growth or viability, genes that represent markers of cell metabolism and stress response (ACTB, GAPDH, p21, BAX) were also simultaneously monitored. To monitor effects on RIG-I-dependent IFN induction, A549 cells were treated with a range of RIG012 concentrations and then the cells were challenged with the RIG-I-specific RNA ligand SLR14 to trigger formation of the activated complex and initiate the IFN signaling pathway. Consistent with the biochemical studies, a potent, dose-dependent effect of RIG012 was observed on both IFN-β and ISG hRsad2 expression (FIG. 3C), indicating that compounds in the RIG012 series are functional inhibitors of endogenous RIG-I signaling in cells. Importantly, genes involved in cell viability and metabolism are unaffected (FIGS. 4A-4B).

The present compound screen, followed by SAR and limited optimization, has yielded specific, high affinity compounds that modulate RIG-I activity in biophysical and cell-based assays. This proof of concept establishes that the RIG-I-like receptors can be targeted and controlled using standard approaches for exploring and optimizing chemical space. This paves the way for a new generation of potent immunomodulatory compounds that can be used as tools for mapping immunological pathways, and as therapeutics for treating autoimmunity and inflammatory diseases that represent areas of unmet medical need.

TABLE 1
Structure-activity relationship analysis of RIG001 and derivatives. Structures and
IC₅₀ values of RIG001 and analogues. For the compounds in the Table, R⁴ and R⁵ are absent.

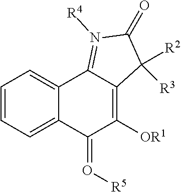

| | R¹ | R² | R³ | IC₅₀ (μM)^{a} |
|---|---|---|---|---|
| RIG001 | —H | —CH₃ | —CH₃ | 12 ± 1 |
| RIG002 | —CH₃ | —CH₃ | —CH₃ | 6.3 ± 0.6 |
| RIG011 | —H | —CH₂CH₃ | —CH₂CH₃ | 0.89 ± 0.04 |
| RIG009 | —H | —CH₂CH₂CH₂CH₂CH₂— | | 2.4 ± 0.3 |
| RIG007 | —CH₂CH₃ | —CH₃ | —CH₃ | 1.8 ± 0.1 |
| RIG006 | 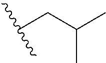 [SMILES: *CC(C)C] | —CH₃ | —CH₃ | 2.1 ± 0.2 |
| RIG005 | 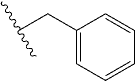 [SMILES: *Cc1ccccc1] | —CH₃ | —CH₃ | 2.6 ± 0.2 |
| RIG013 | 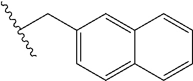 [SMILES: *Cc1ccc2ccccc2c1] | —CH₃ | —CH₃ | 2.1 ± 0.2 |
| RIG008 | 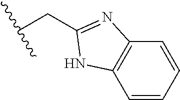 [SMILES: *Cc1nc2ccccc2[nH]1] | —CH₃ | —CH₃ | 1.8 ± 0.3 |
| RIG012 | 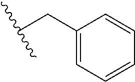 [SMILES: *Cc1ccccc1] | —CH₂CH₃ | —CH₂CH₃ | 0.71 ± 0.02 |

| | | | |
|---|---|---|---|
| RIG010 | 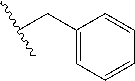 [SMILES: *Cc1ccccc1] | —CH₂CH₂CH₂CH₂CH₂— | 4.7 ± 0.3 |

^{a}The IC₅₀ values are determined using the NADH-coupled ATPase assay and shown as the mean ± standard deviation based on 3 individual measurements.

### Example 4: Compound Synthesis

### 4-Hydroxy-3,3-dimethyl-2H1,3H,5H1-benzo[g]indole-2,5-dione (RIG001)

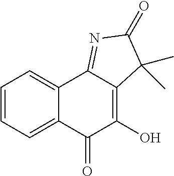 [SMILES: CC1(C)C(=O)N=C2C1=C(O)C(=O)c1ccccc12]

Compound was prepared according to literature procedures (Petersen, et al., 1972, Liebigs Ann. Chem. 764:50; Bystrom, et al., WO 200226707 A1 (2002)).

### 4,5-Dimethoxy-1,3,3-trimethyl-1H,2H,3H-benzo[g]indol-2-one (RIG003)

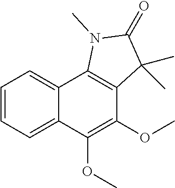 [SMILES: COc1c2c(c3ccccc3c1OC)N(C)C(=O)C2(C)C]

### and 4,5-Dimethoxy-3,3-dimethyl-1H,2H,3H-benzo[g]indol-2-one (RIG004)

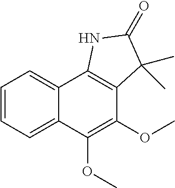 [SMILES: COc1c2c(c3ccccc3c1OC)NC(=O)C2(C)C]

A cooled (3° C.) stirred deep red solution of 4-hydroxy-3,3-dimethyl-2H,3H,5H-benzo[g]indole-2,5-dione (243 mg, 1.0 mmol) in ethanol (5 mL) was treated with sodium borohydride (90 mg) and allowed to reach room temperature over 15 min (became yellow). The mixture was recooled (3° C.) and treated with 25% sodium hydroxide (0.64 g, 4 mmol), then dimethyl sulfate (0.38 mL), and allowed to warm to room temperature and stirred 45 min (started to become red again). Additional sodium borohydride (30 mg) and dimethyl sulfate (0.19 mL) were added with additional ethanol (5 mL), and stirring continued overnight. The reaction was found by TLC to be incomplete, so additional sodium borohydride (60 mg), dimethyl sulfate (0.50 mL), and 25% NaOH (0.50 mL) were added, and stirring continued for another day. The mixture was combined with 10% aqueous citric acid (50 mL) and extracted with methylene chloride (75 mL, then 2×30 mL). The combined organic solution was washed with water and brine (50 mL each), dried (Na₂SO₄), and concentrated in vacuo. The residual material was dissolved in dichloromethane and loaded onto a silica gel column (˜100 cc) and eluted with 10% ethyl acetate/dichloromethane to afford 4,5-dimethoxy-1,3,3-trimethyl-1H,2H,3H-benzo[g]indol-2-one (RIG003, 117 mg, 41%), then 15% ethyl acetate/dichloromethane to afford 4-methoxy-3,3-dimethyl-2H,3H,5Hbenzo[g]indole-2,5-dione (RIG002, 53 mg, 21%, see optimized procedure below) and 4,5-dimethoxy-3,3-dimethyl-1H,2H,3H-benzo[g]indol-2-one (RIG004, 51 mg, 19%).
4,5-Dimethoxy-1,3,3-trimethyl-1H,2H,3H-benzo[g]indol-2-one: ¹H NMR (CDCl₃): δ 8.37 (d, J=7.5 Hz, 1H), 8.14 (d, J=7.5 Hz, 1H), 7.45 (t, J=7.5 Hz, 1H), 7.37 (t, J=7.5 Hz, 1H), 4.06 (s, 3H), 3.93 (s, 3H), 3.77 (s, 3H), 1.50 (s, 6H). ¹³C NMR (CDCl₃): δ 182.88, 146.43, 142.21, 134.11, 129.50, 125.41, 124.51, 122.34, 121.57, 118.68, 60.97, 60.65, 45.13, 30.79, 23.07. LCMS m/z: [M+1]+286.8 (30%).
4,5-Dimethoxy-3,3-dimethyl-1H,2H,3H-benzo[g]indol-2-one: ¹H NMR (CDCl₃): δ 8.10 (m, 1H), 7.80 (m, 1H), 7.47 (m, 2H), 4.08 (s, 3H), 3.95 (s, 3H), 1.50 (s, 6H). ¹³C NMR (CDCl₃): δ 184.87, 146.79, 142.25, 132.06, 128.88, 125.95, 124.90, 124.73, 122.07, 116.94, 61.19, 60.74, 46.84, 23.00. LCMS m/z: [M+1]+272.8 (40%).
4-Methoxy-3,3-dimethyl-2H,3H,5H-benzo[g]indole-2,5-dione (RIG002)

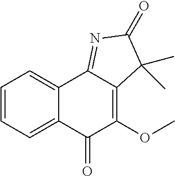 [SMILES: COC1=C2C(=NC(=O)C2(C)C)c2ccccc2C1=O]

An ice cooled stirred solution of 4-hydroxy-3,3-dimethyl-2H,3H,5Hbenzo[g]indole-2,5-dione (241 mg, 1.0 mmol) in THE (15 mL) under nitrogen was treated with 30% methanolic sodium methoxide (216 mg, 1.2 mmol) and stirred for 15 min. Dimethyl sulfate (0.20 mL, 2.11 mmol) was added, the solution allowed to warm to ambient temperature, and the mixture stirred for 20 h. The product was combined with 10% citric acid (15 mL), stirred a few minutes, then extracted with dichloromethane (60 mL, then 30 mL). The combined extracts were washed with saturated bicarbonate and brine (30 mL each), dried (Na₂SO₄), and concentrated in vacuo. The residue was dissolved in dichloromethane, loaded onto a silica gel column (˜80 cc), and eluted with 10% ethyl acetate/dichloromethane to afford 248 mg (97%) of 4-methoxy-3,3-dimethyl-2H,3H,5H-benzo[g]indole-2,5-dione (RIG002) as a dark red solid. ¹H NMR (CDCl₃): δ 8.23 (d, J=7.5 Hz, 1H), 8.03 (d, J=7.5 Hz, 1H), 7.69 (t, J=7.5 Hz, 1H), 7.61 (t, J=7.5 Hz, 1H), 3.70 (s, 3H), 1.50 (s, 6H). ¹³C NMR (d₆-dmso): δ 183.24, 180.45, 172.52, 154.55, 135.22, 132.42, 131.98, 130.43, 127.87, 126.82, 123.41, 43.65, 30.86, 22.66. LCMS m/z: [M+H]⁺ 256.8 (80%).

### 4-Benzyloxy-3,3-dimethyl-2H,3H,5H-benzo[g]indole-2,5-dione (RIG005)

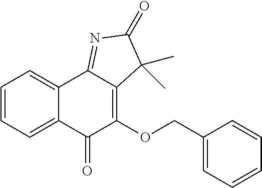 [SMILES: CC1(C)C(=O)N=C2C1=C(OCc1ccccc1)C(=O)c1ccccc12]

An ice cooled stirred solution/suspension of 4-hydroxy-3,3-dimethyl-2H,3H,5Hbenzo[g]indole-2,5-dione (169 mg, 0.7 mmol) in DMF (5 mL) under nitrogen was treated with 60% sodium hydride (30 mg, 0.75 mmol) and warmed to room temperature. Benzyl bromide (0.171 g, 1.0 mmol) was added, the mixture warmed to 65° C., and stirred for 1 h, then cooled to room temperature. The product mixture was quenched with 10% citric acid (10 mL) and extracted with ethyl acetate (40 mL, then 15 mL). The combined organic solution was washed with water and brine (25 mL each), dried (Na₂SO₄), and concentrated in vacuo. The residue was dissolved in minimal dichloromethane, loaded onto a silica gel column (˜75 cc) and eluted with dichloromethane, then 5% ethyl acetate/dichloromethane to afford 185 mg (80%) of 4-benzyloxy-3,3-dimethyl-2H,3H,5H-benzo[g]indole-2,5-dione (RIG005) as a red solid. ¹H NMR (CDCl₃): δ 8.15 (dd, J=8.2 Hz, 1H), 7.64 (d, J=8 Hz, 1H), 7.42-7.52 (m, 2H), 7.37 (t, J=8 Hz, 2H), 7.29 (t, J=7.5 Hz, 1H), 7.17 (d, J=8 Hz, 2H), 5.35 (s, 2H), 1.60 (s, 6H). ¹³C NMR (CDCl₃): δ 183.95, 180.08, 173.00, 153.66, 135.46, 134.54, 132.07, 131.51, 131.11, 129.35, 128.00, 127.11, 125.81, 125.47, 124.80, 45.98, 44.24, 22.78. LCMS m/z: [M+Na]⁺354.5 (30%).

### 4-(2,2-Dimethylethoxy)-3,3-dimethyl-2H,3H,5H-benzo[g]indole-2,5-dione (RIG006)

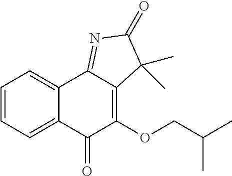 [SMILES: CC(C)COC1=C2C(=NC(=O)C2(C)C)c2ccccc2C1=O]

An stirred solution/suspension of 4-hydroxy-3,3-dimethyl-2H,3H,5H-benzo[g]indole-2,5-dione (121 mg, 0.5 mmol) in DMF (3 mL) under nitrogen was treated with cesium carbonate (326 mg, 1.0 mmol), then 1-iodo-2-methylpropane (0.12 mL, 1.0 mmol) was added, the mixture warmed to 65° C., and stirred for 40 h, then cooled to room temperature. The product mixture was quenched with 10% citric acid (10 mL) and extracted with ethyl acetate (40 mL, then 15 mL). The combined organic solution was washed with water and brine (25 mL each), dried (Na₂SO₄), and concentrated in vacuo. The residue was dissolved in minimal dichloromethane, loaded onto a silica gel column (˜75 cc) and eluted with dichloromethane, then 5% ethyl acetate/dichloromethane to afford 95 mg (64%) of 4-(2,2-dimethylethoxy)-3,3-dimethyl-2H,3H,5H-benzo[g]indole-2,5-dione (RIG006) as a red solid. ¹H NMR (CDCl₃): δ 8.21 (d, J=7.5 Hz, 1H), 7.81 (d, J=7.5 Hz, 1H), 7.68 (t, J=7.5 Hz, 1H), 7.59 (t, J=7.5 Hz, 1H), 3.97 (d, J=7 Hz, 2H), 2.08 (m, 1H), 1.49 (s, 6H), 0.97 (d, J=7 Hz, 6H). ¹³C NMR (CDCl₃): δ 184.03, 180.15, 172.78, 153.21, 134.59, 132.37, 131.58, 131.21, 127.88, 125.07, 125.03, 49.04, 44.041, 29.16, 22.78, 19.74. LCMS m/z: [M+H]⁺ 298.4 (3%); [M+Na]⁺320.5 (4%).

### 4-Ethoxy-3,3-dimethyl-2H,3H,5H-benzo[g]indole-2,5-dione (RIG007)

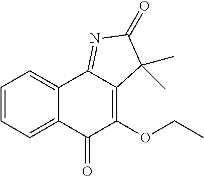 [SMILES: CCOC1=C2C(=NC(=O)C2(C)C)c2ccccc2C1=O]

A stirred solution/suspension of 4-hydroxy-3,3-dimethyl-2H,3H,5H-benzo[g]indole-2,5-dione (241 mg, 1.0 mmol) in DMF (5 mL) under nitrogen was treated with cesium carbonate (652 mg, 2.0 mmol), then iodoethane (0.20 mL, 2.5 mmol) was added, the mixture warmed to 55° C., and stirred for 40 h, then cooled to room temperature. The product mixture was quenched with 10% citric acid (10 mL) and extracted with ethyl acetate (3×50 mL) which contained a small amount of dichloromethane. The combined organic solution was washed with water and brine (25 mL each), dried (MgSO₄), and concentrated in vacuo. The residue was dissolved in dichloromethane and loaded onto a silica gel column (˜75 cc), then eluted with dichloromethane followed by 5% ethyl acetate/dichloromethane to afford 223 mg (83%) of 4-ethoxy-3,3-dimethyl-2H,3H,5H-benzo[g]indole-2,5-dione (RIG007) as a red solid. ¹H NMR (CDCl₃): δ 8.22 (d, J=7.5 Hz, 1H), 7.87 (d, J=7.5 Hz, 1H), 7.70 (t, J=7.5 Hz, 1H), 7.60 (t, J=7.5 Hz, 1H), 4.18 (q, J=7.5 Hz, 2H), 1.48 (s, 6H), 1.43 (t, J=7.5 Hz, 3H). ¹³C NMR (CDCl₃): δ 183.64, 180.19, 172.71, 153.25, 134.70, 132.33, 131.62, 131.25, 127.66, 124.98, 124.83, 44.06, 37.70, 22.51, 14.78. LCMS m/z: [M+H]⁺270.4 (10%).

### 4-(1H-1,3-Benzodiazol-2-ylmethoxy)-3,3-dimethyl-2H,3H,5H-benzo[g]indole-2,5-dione (RIG008)

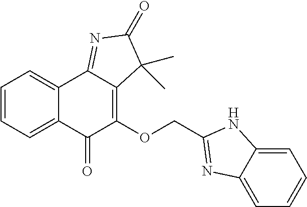 [SMILES: CC1(C)C(=O)N=C2C1=C(OCc1nc3ccccc3[nH]1)C(=O)c1ccccc12]

A stirred solution/suspension of 4-hydroxy-3,3-dimethyl-2H,3H,5H-benzo[g]indole-2,5-dione (241 mg, 1.0 mmol) in DMF (5 mL) under nitrogen was treated with cesium carbonate (652 mg, 2.0 mmol), then 2-chloromethylbenzimidazole (333 mg, 2 mmol) was added, the mixture warmed to 80° C., and stirred for 4 h, then cooled to room temperature. The reaction was quenched with pH 7.4 phosphate buffer (30 mL) and the pH readjusted to ˜7 with 10% aqueous citric acid, then extracted with ethyl acetate (3×75 mL). The combined organic solution was washed with water and brine (50 mL each), dried (MgSO₄) and concentrated in vacuo. The residual red solid was dissolved (mostly) in dichloromethane and added to a silica gel column (˜75 cc) and eluted with 4:1 dichloromethane/ethyl acetate to afford recovered starting material (55 mg), then with 3:1 dichloromethane/ethyl acetate to afford 4-(1H-1,3-benzodiazol-2-ylmethoxy)-3,3-dimethyl-2H,3H,5H-benzo[g]indole-2,5-dione (RIG008) (77 mg, 21% unadjusted for SM) as a red solid. 1H NMR (d6-DMSO): δ 8.00 (m, 2H), 7.53 (m, 2H), 7.46 (m, 2H), 7.12 (m, 2H), 5.53 (br s, 2H), 1.45 (s, 6H). ¹³C NMR (d6-DMSO): δ 183.89, 180.13, 173.10, 153.86, 149.43, 135.07, 132.09, 131.73, 130.45, 127.36, 126.36, 124.20, 122.53, 43.81, 41.18, 22.64. LCMS m/z: [M+H]⁺372.6 (1%).

### 4-Hydroxy-2,5-dihydrospiro[benzo[g]indole-3,1′-cyclohexane]-2,5-dione (RIG009)

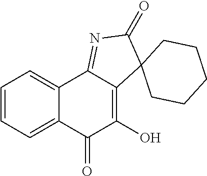 [SMILES: O=C1C(O)=C2C(=NC(=O)C23CCCCC3)c2ccccc21]

A stirred suspension of 1,4-naphthoquinone (5.22 g, 33 mmol) in methanol (25 mL) under nitrogen was treated dropwise with 4-(cyclohexylidenemethyl)morpholine (7.0 g, 38.6 mmol) and heated to 50° C. After 1 h the reaction was incomplete by TLC (DCM as eluent), and additional 4-(cyclohexylidenemethyl)morpholine (1.5 g, 8.3 mmol) added. After another hour a thick precipitate formed, and this was cooled and diluted with water (15 mL). The suspension was filtered and the solid rinsed with 2:1 methanol/water, collected and dried in vacuo at 50° C. The solid was suspended in water (25 mL), treated with FeCl₃.6H₂O (18 g, 66 mmol) dissolved in water (25 mL), and stirred for 3 h. The suspension was filtered, the solid rinsed with water, collected and dried in vacuo. The solid was dissolved in dichloromethane, loaded onto a pad of silica gel (˜200 cc) and eluted with dichloromethane to afford 4.51 g (51%) of 1-(1,4-dioxo-1,4-dihydronaphthalen-2-yl)cyclohexane-1-carboxaldehyde as a green-gray solid. A stirred solution of 1-(1,4-dioxo-1,4-dihydronaphthalen-2-yl)cyclohexane-1-carboxaldehyde (2.68 g, 10 mmol) in methanol (125 mL) and water (12.5 mL) was treated with 50% aqueous hydroxylamine (1.0 mL, 15 mmol) and stirred at room temperature for 3 h, then concentrated in vacuo to a solid. This was taken up in 95% ethanol (90 mL), treated with 6N HCl/isopropanol (25 mL) and heated to 60° C. for 1 h. Benzoquinone (10 g, 92.5 mmol) was added, and heating at 60° C. continued for 1 h. The solution was cooled to room temperature and concentrated in vacuo. The residue was dissolved in 5% ethyl acetate/dichloromethane, loaded onto a silica gel column (˜200 cc) and eluted first with 5% EtOAc/DCM (to remove impurities), then 15% EtOAc/DCM to afford product, which was triturated from ether to give 1.61 g (57%) of 4-hydroxy-2,5-dihydrospiro[benzo[g]indole-3,1′-cyclohexane]-2,5-dione (RIG009) as a red solid. ¹H NMR (CDCl₃): δ 11.17 (br s, 1H), 8.08 (d, J=7.5 Hz, 1H), 7.81 (d, J=7.5 Hz, 1H), 7.64 (t, J=7.5 Hz, 1H), 7.52 (t, J=7.5 Hz, 1H), 2.14-2.26 (m, 2H), 1.90-2.05 (m, 2H), 1.60-1.80 (m, 5H), 1.35-1.50 (m, 1H). ¹³C NMR (CDCl₃): δ 183.98, 180.75, 172.25, 154.35, 134.83, 131.93, 131.66, 129.75, 127.00, 125.26, 122.56, 48.91, 30.39, 24.77, 20.33. LCMS m/z: [M+H]⁺282.4 (10%); [M−H]⁻ 280.5 (100%).

### 4-(Benzyloxy)-2,5-dihydrospiro[benzo[g]indole-3,1′-cyclohexane]-2,5-dione (RIG010)

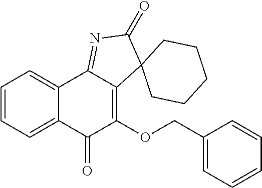 [SMILES: O=C1C(OCc2ccccc2)=C2C(=NC(=O)C23CCCCC3)c2ccccc21]

A stirred solution of 4-(hydroxy)-2,5-dihydrospiro[benzo[g]indole-3,1′-cyclohexane]-2,5-dione (281 mg, 1.0 mmol) in anhydrous DMF (5 mL) under nitrogen was treated with cesium carbonate (500 mg, 1.53 mmol) and benzyl bromide (0.18 mL, 1.5 mmol), heated to 65° C. for 4 h, then cooled to room temperature and combined with 10% citric acid (10 mL). The mixture was extracted with ether (30 mL, then 2×10 mL) and the combined solution washed with water and brine (20 mL each), dried (MgSO₄) and concentrated in vacuo. The residual red solid was dissolved in dichloromethane and loaded onto a silica gel column (˜80 cc) and eluted with DCM, then 3% EtOAc/DCM to afford 304 mg (82%) of 4-(benzyloxy)-2,5-dihydrospiro[benzo[g]indole-3,1′-cyclohexane]-2,5-dione (RIG010) as a red solid. ¹H NMR (CDCl₃): δ 8.13 (dd, J=7.5.2 Hz, 1H), 7.63 (d, J=7.5 Hz, 1H), 7.33-7.50 (m, 4H), 7.28 (t, J=7.5 Hz, 1H), 7.17 (d, J=7.5 Hz, 2H), 5.32 (s, 2H), 2.32-2.47 (m, 2H), 2.00-2.15 (m, 2H), 1.81 (m, 1H), 1.60-1.75 (m, 4H), 1.45 (m, 1H). ¹³C NMR (CDCl₃): δ 182.22, 180.12, 172.81, 154.01, 135.67, 134.48, 132.22, 131.39, 130.94, 129.32, 127.90, 127.15, 125.82, 125.48, 123.74, 47.19, 45.59, 30.40, 24.69, 20.29. LCMS m/z: [M+Na]+ 394.6 (40%).

### 3,3-Diethyl-4-hydroxy-2H,3H,5H-benzo[g]indole-2,5-dione (RIG011)

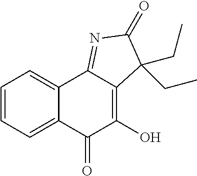 [SMILES: CCC1(CC)C(=O)N=C2C1=C(O)C(=O)c1ccccc12]

A stirred suspension of 1,4-naphthoquinone (5.22 g, 33 mmol) in methanol (25 mL) under nitrogen was treated dropwise with 4-(2-ethylbut-1-en-1-yl)morpholine (7.62 g, 45 mmol) and heated to 50° C. for 3 h. The mixture was cooled and diluted with water (20 mL). The suspension was filtered and the solid rinsed with 2:1 methanol/water, collected and dried in vacuo at 50° C. The solid was suspended in water (25 mL), treated with FeCl₃.6H₂O (18 g, 66 mmol) dissolved in water (25 mL), and stirred overnight at room temperature. The suspension was filtered, the solid rinsed with water, collected and dried in vacuo. The solid was dissolved in dichloromethane, loaded onto a pad of silica gel (˜200 cc) and eluted with dichloromethane to afford 4.60 g (54%) of 2-(1,4-dioxo-1,4-dihydronaphthalen-2-yl)-2-ethylbutanal as a green-gray solid. A stirred solution of 2-(1,4-dioxo-1,4-dihydronaphthalen-2-yl)-2-ethylbutanal (2.56 g, 10 mmol) in methanol (125 mL) and water (12.5 mL) was treated with 50% aqueous hydroxylamine (1.0 mL, 15 mmol) and stirred at room temperature for 3 h, then concentrated in vacuo to a solid. This was taken up in 95% ethanol (90 mL), treated with 6N HCl/isopropanol (25 mL) and heated to 60° C. for 1 h. Benzoquinone (10 g, 92.5 mmol) was added, and heating at 60° C. continued for 1 h. The solution was cooled to room temperature and concentrated in vacuo. The residue was dissolved in 5% ethyl acetate/dichloromethane, loaded onto a silica gel column (˜200 cc) and eluted first with 5% EtOAc/DCM (to remove impurities), then 15% EtOAc/DCM to afford product, which was triturated from ether to give 2.04 g (76%) of 3,3-diethyl-4-hydroxy-2H,3H,5H-benzo[g]indole-2,5-dione (RIG011) as a red solid. ¹H NMR (CDCl₃): δ 11.46 (br s, 1H), 8.04 (d, J=7.5 Hz, 1H), 7.78 (d, J=7.5 Hz, 1H), 7.60 (t, J=7.5 Hz, 1H), 7.50 (t, J=7.5 Hz, 1H), 2.04 (m, 2H), 1.77 (m, 2H), 0.67 (t, J=7.5 Hz, 6H). ¹³C NMR (CDCl₃): δ 183.90, 180.77, 172.35, 156.56, 149.86, 134.90, 131.77, 129.75, 126.76, 125.40, 115.95, 56.64, 29.03, 9.15. LCMS m/z: [M+H]⁺270.4 (5%); [M−H]⁻ 268.4 (30%).

### 4-Benzyloxy-3,3-diethyl-2H,3H,5H-benzo[g]indole-2,5-dione (RIG012)

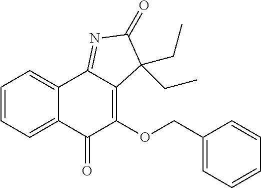 [SMILES: CCC1(CC)C(=O)N=C2C1=C(OCc1ccccc1)C(=O)c1ccccc12]

A stirred solution of 3,3-diethyl-4-hydroxy-2H,3H,5H-benzo[g]indole-2,5-dione (269 mg, 1.0 mmol) in anhydrous DMF (5 mL) under nitrogen was treated with cesium carbonate (500 mg, 1.53 mmol) and benzyl bromide (0.18 mL, 1.5 mmol), heated to 65° C. for 4 h, then cooled to room temperature and combined with 10% citric acid (10 mL). The mixture was extracted with ether (30 mL, then 2×10 mL) and the combined solution washed with water and brine (20 mL each), dried (MgSO₄) and concentrated in vacuo. The residual red solid was dissolved in dichloromethane and loaded onto a silica gel column (˜80 cc) and eluted with DCM, then 3% EtOAc/DCM to afford 208 mg (58%) of 4-benzyloxy-3,3-diethyl-2H,3H,5H-benzo[g]indole-2,5-dione (RIG013) as a red solid. ¹H NMR (CDCl₃): δ 8.18 (d, J=7.5 Hz, 1H), 7.62 (d, J=7.5 Hz, 1H), 7.50 (t, J=7.5 Hz, 1H), 7.45 (t, J=7.5 Hz, 1H), 7.37 (m, 2H), 7.31 (m, 1H), 7.23 (m, 2H), 5.34 (br s, 2H), 2.25 (m, 2H), 1.97 (m, 2H), 0.78 (t, J=7.5 Hz, 6H). ¹³C NMR (CDCl₃): δ 182.94, 180.06, 172.87, 156.14, 135.47, 134.60, 132.29, 131.55, 131.13, 129.29, 127.95, 125.63, 55.21, 46.25, 29.51, 9.43. LCMS m/z: [M+Na]⁺382.6 (30%).

### 3,3-Dimethyl-4-(naphthalen-2-ylmethoxy)-2H,3H,5H-benzo[g]indole-2,5-dione (RIG013)

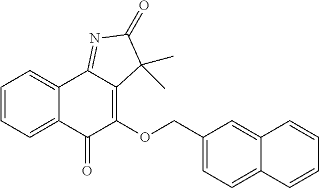 [SMILES: CC1(C)C(=O)N=C2C1=C(OCc1ccc3ccccc3c1)C(=O)c1ccccc12]

A stirred solution of 4-hydroxy-3,3-dimethyl-2H,3H,5H-benzo[g]indole-2,5-dione (241 mg, 1.0 mmol) in anhydrous DMF (5 mL) under nitrogen was treated with cesium carbonate (500 mg, 1.53 mmol) and 2-naphthylmethyl bromide (332 mg, 1.5 mmol), heated to 65° C. for 4 h, then cooled to room temperature and combined with 2% citric acid (25 mL). The suspension was filtered, the solid rinsed with water, partially air dried, and dissolved in dichloromethane (50 mL) and dried (Na₂SO₄). The dried solution was added directly to a silica gel column (˜100 cc) and eluted with dichloromethane, then 2.5% EtOAc/DCM, then 5% EtOAc/DCM to afford 257 mg (67%) of 3,3-dimethyl-4-(naphthalen-2-ylmethoxy)-2H,3H,5Hbenzo[g]indole-2,5-dione (RIG013) as a red solid. ¹H NMR (CDCl₃): δ 8.14 (dd, J=7.5, 1.5 Hz, 1H), 7.88 (d, J=8 Hz, 1H), 7.83 (m, 1H), 7.76 (m, 1H), 7.68 (d, J=8 Hz, 1H), 7.56 (s, 1H), 7.48 (m, 2H), 7.44 (td, J=8.1 Hz, 1H), 7.38 (td, J=8, 1.5 Hz, 1H), 7.32 (dd, J=8, 1.5 Hz, 1H), 5.50 (s, 2H), 1.65 (s, 6H). ¹³C NMR (CDCl₃): δ 184.00, 180.08, 173.06, 153.71, 134.61, 133.35, 132.95, 132.85, 132.08, 131.50, 131.10, 129.48, 127.80, 127.76, 127.10, 126.78, 126.40, 125.79, 124.84, 124.24, 123.32, 46.19, 44.32, 22.85. LCMS m/z: [M+Na]⁺404.7 (5%).

### Enumerated Embodiments

The following exemplary embodiments are provided, the numbering of which is not to be construed as designating levels of importance.

Embodiment 1 provides a compound of formula (I), or a salt, solvate, isotopically labelled derivative, stereoisomer, tautomer, or geometric isomer thereof:

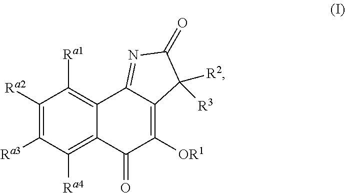

wherein: R¹ is selected from the group consisting of H, optionally substituted C₁-C₆ alkyl, optionally substituted C₂-C₆ alkenyl, optionally substituted C₂-C₆ alkynyl, optionally substituted C₃-C₅ cycloalkyl, optionally substituted —(C₀-C₆ alkylene)-phenyl, optionally substituted —(C₀-C₆ alkylene)-naphthyl, and optionally —(C₀-C₆ alkylene)-substituted heteroaryl; R² is selected from the group consisting of H, optionally substituted C₁-C₆ alkyl, optionally substituted C₂-C₆ alkenyl, optionally substituted C₂-C₆ alkynyl, and optionally substituted C₃-C₅ cycloalkyl; R³ is selected from the group consisting of H, optionally substituted C₁-C₆ alkyl, optionally substituted C₂-C₆ alkenyl, optionally substituted C₂-C₆ alkynyl, and optionally substituted C₃-C₅ cycloalkyl; or R² and R³ combine to form optionally substituted C₂-C₇ alkylene; each occurrence of R^{a1}, R^{a2}, R^{a3}, and R^{a4} is independently selected from the group consisting of H, F, Cl, Br, I, CN, C₁-C₆ alkyl, C₃-C₈ cycloalkyl, phenyl, C₁-C₆ hydroxyalkyl, C₁-C₆ haloalkyl, C₁-C₆ haloalkoxy, (C₁-C₆ alkoxy)-C₀-C₆ alkylene, —NR^{b}R^{b}, —OR^{b}, —C(═O)OR^{b}, and —C(═O)N(R^{b})(R^{b}), wherein each occurrence of R^{b} is independently H, C₁-C₆ alkyl, or C₃-C₈ cycloalkyl; and n is 0, 1, 2, 3, or 4.

Embodiment 2 provides the compound of Embodiment 1, wherein R¹ is H, methyl, ethyl, isobutyl, benzyl, —CH₂-naphthyl, or —CH₂—(2-benzimidazolyl).

Embodiment 3 provides the compound of any of Embodiments 1-2, wherein R² is H, methyl, or ethyl.

Embodiment 4 provides the compound of any of Embodiments 1-3, wherein R³ is H, methyl, or ethyl.

Embodiment 5 provides the compound of any of Embodiments 12, wherein R² and R³ combine to form —CH₂—, —(CH₂)₂—, —(CH₂)₃—, —(CH₂)₄—, —(CH₂)₅—, —(CH₂)₆—, or —(CH₂)₇—.

Embodiment 6 provides the compound of any of Embodiments 1-5, wherein the compound of formula (I) is selected from the group consisting of: 4-Hydroxy-3,3-dimethyl-2H,3H,5H-benzo[g]indole-2,5-dione; 4-Methoxy-3,3-dimethyl-2H,3H,5H-benzo[g]indole-2,5-dione; 4-Benzyloxy-3,3-dimethyl-2H,3H,5H-benzo[g]indole-2,5-dione; 4-(2,2-Dimethylethoxy)-3,3-dimethyl-2H,3H,5H-benzo[g]indole-2,5-dione; 4-Ethoxy-3,3-dimethyl-2H,3H,5H-benzo[g]indole-2,5-dione; 4-(1H-1,3-Benzodiazol-2-ylmethoxy)-3,3-dimethyl-2H,3H,5H-benzo[g]indole-2,5-dione; 4-Hydroxy-2,5-dihydrospiro[benzo[g]indole-3,1′-cyclohexane]-2,5-dione; 4-(Benzyloxy)-2,5-dihydrospiro[benzo[g]indole-3,1′-cyclohexane]-2,5-dione; 3,3-Diethyl-4-hydroxy-2H,3H,5H-benzo[g]indole-2,5-dione; 4-Benzyloxy-3,3-diethyl-2H,3H,5H-benzo[g]indole-2,5-dione; and 3,3-Dimethyl-4-(naphthalen-2-ylmethoxy)-2H,3H,5H-benzo[g]indole-2,5-dione.

Embodiment 7 provides a pharmaceutical composition comprising at least one compound of any of Embodiments 1-6.

Embodiment 8 provides a method of inhibiting RIG-I activity in a subject, the method comprising administering to the subject a therapeutically effective amount of at least one compound of any of Embodiments 1-6.

Embodiment 9 provides a method of treating, ameliorating, and/or preventing a disease or disorder associated with defective distribution and processing of host RNA molecules in a subject, the method comprising administering to the subject a therapeutically effective amount of at least one compound of any of Embodiments 1-6.

Embodiment 10 provides a method of treating, ameliorating, and/or preventing a disease or disorder associated with malfunction of the human RNA decay machinery in a subject, the method comprising administering to the subject a therapeutically effective amount of at least one compound of any of Embodiments 1-6.

Embodiment 11 provides a method of treating, ameliorating, and/or preventing an autoimmune disorder in a subject, the method comprising administering to the subject a therapeutically effective amount of at least one compound of any of Embodiments 1-6.

Embodiment 12 provides the method of Embodiment 11, wherein the autoimmune disorder comprises type-I diabetes and/or Sjögren's syndrome.

Embodiment 13 provides a method of treating, ameliorating, and/or preventing COPD in a subject, the method comprising administering to the subject a therapeutically effective amount of at least one compound of any of Embodiments 1-6.

Embodiment 14 provides a method of treating, ameliorating, and/or preventing an inflammatory disease in a subject, the method comprising administering to the subject a therapeutically effective amount of at least one compound of any of Embodiments 1-6.

Embodiment 15 provides the method of any of Embodiments 8-14, wherein the at least one compound is administered as part of a pharmaceutical composition.

Embodiment 16 provides the method of any of Embodiments 8-15, wherein the subject is further administered another therapeutic agent that treats, ameliorates, and/or prevents the disease or disorder.

Embodiment 17 provides the method of any of Embodiments 8-16, wherein the subject is a mammal.

Embodiment 18 provides the method of any of Embodiments 8-17, wherein the subject is a human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A compound of formula (I), or a salt, solvate, isotopically labelled derivative, stereoisomer, tautomer, or geometric isomer thereof: 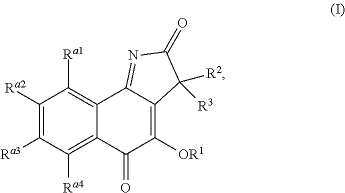
wherein:
R¹ is selected from the group consisting of H, optionally substituted C₁-C₆ alkyl, optionally substituted C₂-C₆ alkenyl, optionally substituted C₂-C₆ alkynyl, optionally substituted C₃-C₈ cycloalkyl, optionally substituted —(C₀-C₆ alkylene)-phenyl, optionally substituted —(C₀-C₆ alkylene)-naphthyl, and optionally —(C₀-C₆ alkylene)-substituted heteroaryl;
R² is selected from the group consisting of H, optionally substituted C₁-C₆ alkyl, optionally substituted C₂-C₆ alkenyl, optionally substituted C₂-C₆ alkynyl, and optionally substituted C₃-C₈ cycloalkyl;
R³ is selected from the group consisting of H, optionally substituted C₁-C₆ alkyl, optionally substituted C₂-C₆ alkenyl, optionally substituted C₂-C₆ alkynyl, and optionally substituted C₃-C₈ cycloalkyl;
or R² and R³ combine to form optionally substituted C₂-C₇ alkylene;
each occurrence of R^{a1}, R^{a2}, Ras, and R^{a4} is independently selected from the group consisting of H, F, Cl, Br, I, CN, C₁-C₆ alkyl, C₃-C₈ cycloalkyl, phenyl, C₁-C₆ hydroxyalkyl, C₁-C₆ haloalkyl, C₁-C₆ haloalkoxy, (C₁-C₆ alkoxy)-C₀-C₆ alkylene, —NR^{b}R^{b}, —OR, —C(═O)OR^{b}, and —C(═O)N(R^{b})(R^{b}),
wherein each occurrence of R^{b} is independently H, C₁-C₆ alkyl, or C₃-C₈ cycloalkyl.

2. The compound of claim 1, wherein R¹ is H, methyl, ethyl, isobutyl, benzyl, —CH₂-naphthyl, or —CH₂—(2-benzimidazolyl).

3. The compound of claim 1, wherein R² is H, methyl, or ethyl.

4. The compound of claim 1, wherein R³ is H, methyl, or ethyl.

5. The compound of claim 1, wherein R² and R³ combine to form —CH₂—, —(CH₂)₂—, —(CH₂)₃—, —(CH₂)₄—, —(CH₂)₅—, —(CH₂)₆—, or —(CH₂)₇—.

6. The compound of claim 1, which is selected from the group consisting of:
4-Hydroxy-3,3-dimethyl-2H,3H,5H-benzo[g]indole-2,5-dione 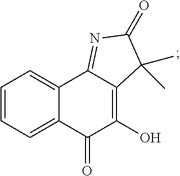 [SMILES: CC1(C)C(=O)N=C2C1=C(O)C(=O)c1ccccc12]
4-Methoxy-3,3-dimethyl-2H,3H,5H-benzo[g]indole-2,5-dione 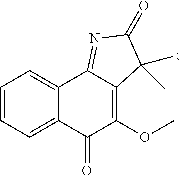 [SMILES: COC1=C2C(=NC(=O)C2(C)C)c2ccccc2C1=O]
4-Benzyloxy-3,3-dimethyl-2H,3H,5H-benzo[g]indole-2,5-dione 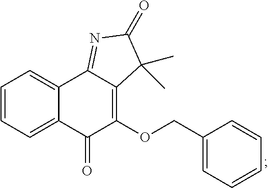 [SMILES: CC1(C)C(=O)N=C2C1=C(OCc1ccccc1)C(=O)c1ccccc12]
4-(2,2-Dimethylethoxy)-3,3-dimethyl-2H,3H,5H-benzo[g]indole-2,5-dione 0 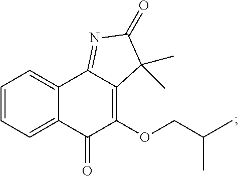 [SMILES: CC(C)COC1=C2C(=NC(=O)C2(C)C)c2ccccc2C1=O]
4-Ethoxy-3,3-dimethyl-2H,3H,5H-benzo[g]indole-2,5-dione 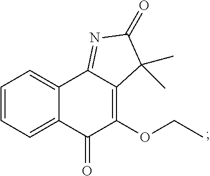 [SMILES: CCOC1=C2C(=NC(=O)C2(C)C)c2ccccc2C1=O]
4-(1H-1,3-Benzodiazol-2-ylmethoxy)-3,3-dimethyl-2H,3H,5H-benzo[g]indole-2,5-dione 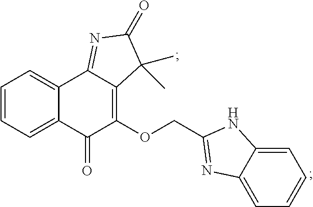 [SMILES: CC1(C)C(=O)N=C2C1=C(OCc1nc3ccccc3[nH]1)C(=O)c1ccccc12]
4-Hydroxy-2,5-dihydrospiro[benzo[g]indole-3,1′-cyclohexane]-2,5-dione 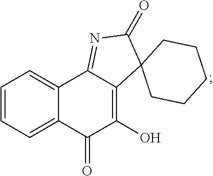 [SMILES: O=C1C(O)=C2C(=NC(=O)C23CCCCC3)c2ccccc21]
4-(Benzyloxy)-2,5-dihydrospiro[benzo[g]indole-3,1′-cyclohexane]-2,5-dione 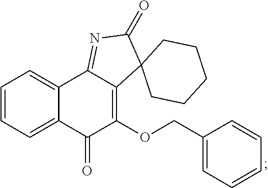 [SMILES: O=C1C(OCc2ccccc2)=C2C(=NC(=O)C23CCCCC3)c2ccccc21]
3,3-Diethyl-4-hydroxy-2H,3H,5H-benzo[g]indole-2,5-dione 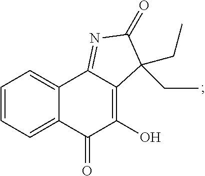 [SMILES: CCC1(CC)C(=O)N=C2C1=C(O)C(=O)c1ccccc12]
4-Benzyloxy-3,3-diethyl-2H,3H,5H-benzo[g]indole-2,5-dione 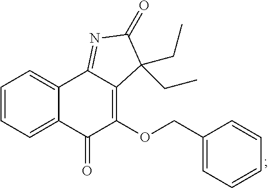 [SMILES: CCC1(CC)C(=O)N=C2C1=C(OCc1ccccc1)C(=O)c1ccccc12]
and
3,3-Dimethyl-4-(naphthalen-2-ylmethoxy)-2H,3H,5H-benzo[g]indole-2,5-dione

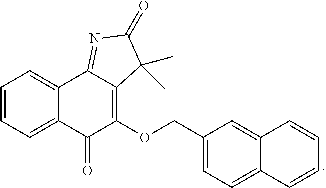 [SMILES: CC1(C)C(=O)N=C2C1=C(OCc1ccc3ccccc3c1)C(=O)c1ccccc12]

7. A pharmaceutical composition comprising at least one compound claim 1.

8. A method of inhibiting RIG-I activity in a subject, the method comprising administering to the subject a therapeutically effective amount of at least one compound of claim 1.

9. A method of treating, ameliorating, or preventing a disease or disorder associated with defective distribution and processing of host RNA molecules in a subject, the method comprising administering to the subject a therapeutically effective amount of at least one compound of claim 1, which is optionally administered as part of a pharmaceutical composition.

10. A method of treating, ameliorating, or preventing a disease or disorder associated with malfunction of the human RNA decay machinery in a subject, the method comprising administering to the subject a therapeutically effective amount of at least one compound of claim 1, which is optionally administered as part of a pharmaceutical composition.

11. A method of treating, ameliorating, or preventing an autoimmune disorder in a subject, the method comprising administering to the subject a therapeutically effective amount of at least one compound of claim 1, which is optionally administered as part of a pharmaceutical composition.

12. The method of claim 11, wherein the autoimmune disorder comprises type-I diabetes or Sjögren's syndrome.

13. A method of treating, ameliorating, or preventing COPD in a subject, the method comprising administering to the subject a therapeutically effective amount of at least one compound of claim 1, which is optionally administered as part of a pharmaceutical composition.

14. A method of treating, ameliorating, or preventing an inflammatory disease in a subject, the method comprising administering to the subject a therapeutically effective amount of at least one compound of claim 1, which is optionally administered as part of a pharmaceutical composition.

15. (canceled)

16. The method of claim 9, wherein the subject is further administered another therapeutic agent that treats, ameliorates, or prevents the disease or disorder.

17. The method of claim 9, wherein the subject is a mammal.

18. The method of claim 9, wherein the subject is a human.


SEQUENCE LISTING (12 sequences)

SEQ ID NO: 1 - DNA, 23 bp, Artificial Sequence
  Chemically synthesized
tggtcaggcagtataatccaaag

SEQ ID NO: 2 - DNA, 23 bp, Artificial Sequence
  Chemically synthesized
tttcaaatccaacaaagtctggc

SEQ ID NO: 3 - DNA, 20 bp, Artificial Sequence
  Chemically synthesized
gcaagagcacaagaggaaga

SEQ ID NO: 4 - DNA, 20 bp, Artificial Sequence
  Chemically synthesized
ctacatggcaactgtgagga

SEQ ID NO: 5 - DNA, 20 bp, Artificial Sequence
  Chemically synthesized
ttccagcagatgtggatcag

SEQ ID NO: 6 - DNA, 20 bp, Artificial Sequence
  Chemically synthesized
ggtgtaacgcaactaagtca

SEQ ID NO: 7 - DNA, 18 bp, Artificial Sequence
  Chemically synthesized
tgcccaagctctaccttc

SEQ ID NO: 8 - DNA, 19 bp, Artificial Sequence
  Chemically synthesized
gacagtgacaggtccacat

SEQ ID NO: 9 - DNA, 19 bp, Artificial Sequence
  Chemically synthesized
caccagctctgagcagatc

SEQ ID NO: 10 - DNA, 18 bp, Artificial Sequence
  Chemically synthesized
gctgccactcggaaaaag

SEQ ID NO: 11 - DNA, 20 bp, Artificial Sequence
  Chemically synthesized
tcgctatctcctgtgacagc

SEQ ID NO: 12 - DNA, 20 bp, Artificial Sequence
  Chemically synthesized
caccacctcctcagcttttg